(19) United States
(12) Patent Application Publication
(10) Pub. No.: US 2014/0359795 A1
(43) Pub. Date: Dec. 4, 2014

(54) GENETIC TECHNIQUES FOR MAKING ANIMALS WITH SORTABLE SPERM

(71) Applicant: Recombinetics, Inc., Saint Paul, MN (US)

(72) Inventors: Scott C. Fahrenkrug, Minneapolis, MN (US); Daniel F. Carlson, Woodbury, MN (US)

(21) Appl. No.: 14/263,431

(22) Filed: Apr. 28, 2014

Related U.S. Application Data
(60) Provisional application No. 61/829,672, filed on May 31, 2013.
(60) Provisional application No. 61/870,586, filed on Aug. 27, 2013.

(51) Int. Cl. A01K 67/027; A61K 35/52

(52) U.S. Cl. CPC A01K 67/0275; A61K 35/52; A01K 2217/052; A01K 2227/10; A01K 2267/02
USPC 800/13; 435/2; 435/283.1

(57) ABSTRACT

Livestock with sperm labeled to indicate an X and/or Y chromosome. Male livestock that produce progeny of only one gender. Sperm that have a marker.

### CROSS-REFERENCE TO RELATED APPLICATIONS

This application claims priority to U.S. Provisional Application Nos. 61/829,672 filed May 31, 2013 and 61/870,586 filed Aug. 27, 2013, each of which are hereby incorporated by reference.

### STATEMENT OF GOVERNMENT SUPPORT

Aspects of the work described herein were supported by Biotechnology Risk Assessment Program competitive grant number 2012-33522-19766 from the USDA—National Institute of Food and Agriculture. The United States Government may have certain rights in these inventions.

### TECHNICAL FIELD

The technical field relates to genetically modified animals, and particularly to animals having sperm that is genetically modified for sorting by gender.

### BACKGROUND

Animal breeding and raising practices would be improved if sperm could be presorted to sort sperm with a Y chromosome gamete from those with an X chromosome gamete. Animals could then be created from the sperm to have a predetermined gender.

### SUMMARY

Founder animals with sperm that are readily sortable are provided. The sperm are suitable for effective ex vivo sorting processes. Embodiments include sperm labeled with a marker. The marker provides visualization and/or a tag for binding. Both positive and negative selection are provided. Ex vivo sorting processes using the sperm include, for example, techniques based on visualization, marker-based sorting, negative selection, e.g., with toxins or assays based on motility. The following patent applications are hereby incorporated herein by reference for all purposes; in case of conflict, the specification is controlling: US 2010/0146655, US 2010/0105140, US 2011/0059160, and US 2011/0197290.

### BRIEF DESCRIPTION OF THE DRAWINGS

FIG. 1 illustrates the use of genetic tools to create animals with genetically modified sperm.

FIG. 2A illustrates a mammalian sperm with modified sites external and/or internal to the sperm.

FIG. 2B illustrates an embodiment for sorting sperm using a solid phase having ligands that bind markers on sperm surfaces.

FIG. 3 depicts creation of animals with sperm marked to indicate gender.

FIG. 4 depicts experimental results for modification of a vertebrate Y chromosome.

FIG. 5 is a montage of experimental results of Examples 6 and 7 showing CRISPR/Cas9 mediated HDR used to introgress the p65 S531 P mutation from warthogs into conventional swine. Panel a) The S531P missense mutation Panel b) SURVEYOR assay of transfected Landrace fibroblasts Panels c and d) show RFLP analysis of cells sampled at days 3 and 10. The top and bottom rows of sequences in panel a are the guide RNA (gRNA) (P65_G1S having SEQ ID NO:1 and P65_G2A having SEQ ID NO:2). The second row is the wildtype (Wt) P65 sequence, SEQ ID NO:3. The third row is the HDR template, SEQ ID NO:4, used in the experiment. The left TALEN (SEQ ID NO:5) and right TALEN, (SEQ ID NO:6) are shown.

FIG. 6 is a montage of experiment results showing a comparison of TALENs and CRISPR/Cas9 mediated HDR at porcine APC. Panel a) depicts the APC14.2 TALENs and the gRNA sequence APC14.2 G1a relative to the wild type APC sequence. Below, the HDR oligo is shown which delivers a 4 bp insertion (underlined text) resulting in a novel HindIII site. Panel b) shows charts displaying RFLP and SURVEYOR assay results. The top row of panel a is the APC14.2 TALENs sequence, SEQ ID NO:7. The second row is the wildtype APCS sequence, SEQ ID NO:8. The third row shows the gRNA sequence G1a, SEQ ID NO:9. The bottom sequence is the HDR template, SEQ ID NO:10.

FIG. 7 shows gene targeting of the vertebrate Y chromosome in two sites (AMELY and SRY) using TALENs and plasmid homology templates. Individual colonies are screened using a locus specific primer outside of the homology arms and a transgene specific primer within the homology template. The locus and orientation of the homology template is indicated above their corresponding wells and positive controls are indicated (+).

FIG. 8 is a Table showing analysis results of Y-targeting in clones with TALENs and plasmid homology cassettes.

FIG. 9 is short homology targeting of Ubiquitin EGPF to 3 sites in the Y-chromosome. Primers for the 3′ junction of SRY also gave a non-specific banding pattern with and without TALENs.

FIG. 10 is a bar graph showing expression of the EGFP marker in cells treated with TALENs and short homology templates specific to AMELY and SRY sites.

FIG. 11 is a junction analysis of clones expressing the EGFP marker.

FIG. 12 provides a general schematic of the cisX vector. Either the swine SP10, ACE, or CK15 promoters were placed upstream of the cisX cassette. This cisX cassette consists of Smok1 5 and 3′ UTRs flanking the EGFP transgene. The entire promoter-transgene cassette is flanked by Sleeping Beauty IRDRs to facilitate for enzymatic insertion of the transgene by provision of a source of transposase, SB100X.

FIG. 13. Top, qPCR for EGFP transcript in mouse testes normalized to Itga6. The founder line is indicated below along with whether the mouse analyzed was an F0 or an F1. The F0 transmission of the transgene is provided giving an estimate of total copy number of the transgene.

FIG. 14. Fluorescent labeling of testis tissue in mice expressing GFP regulated by cis restriction under the SP 10 promoter using antibodies against green fluorescent protein.

FIG. 15. Fluorescent labeling of spermatozoa in male mice expressing GFP regulated by cis restriction under the SP 10 promoter using antibodies against green fluorescent protein.

### DETAILED DESCRIPTION

Animals are provided that have sperm marked to indicate gender of the sex chromosome in the sperm. Highly effective ex vivo sorting processes may be used to separate the marked sperm. Markers may be used for sorting of sperm using imaging of the marker, or the markers may provide a site for binding. Positive and/or negative selection may be used. Ex vivo sorting processes using the sperm include, for example, techniques based on visualization, marker-based sorting, negative selection, e.g., with toxins. Motility-based assays are also available, for instance, with poorly-swimming sperm being separated from wild type-swimming sperm. Embodiments include, for instance, fusion proteins of a marker and a sperm component. Proteins and genes that can be modified to make the fusion protein are detailed herein. Data detailing modifications made to sex chromosomes are included.

### Sperm Anatomy and Formation

A mammalian sperm cell has a head, a midpiece and a tail. The head contains the nucleus with densely coiled chromatin fibres, surrounded anteriorly by an acrosome, which contains enzymes used for penetrating the female egg. The midpiece has a central filamentous core with many mitochondria packed around it, used for ATP production for the journey through the female cervix, uterus and uterine tubes. The tail or “flagellum” executes the lashing movements that propel the spermatocyte.

Spermatogenesis refers to the process by which sperms are formed within the seminiferous tubules from spermatogonia or sperm mother cells which lie on the basement membrane. Spermatogenesis has two distinct phases: spermatocytogenesis, which is a series of divisions during which spermatogonia form spermatids. The second phase is spermatogenesis: a phase where spermatids undergo metamorphosis forming spermatozoa. The entire process takes a number of weeks, for instance, about 60 days in bull and 49 about days in rani.

Spermatocytogenesis has four phases. Phase 1 (generally about 15 days duration) is the mitotic division of spermatogonium. The dormant spermatogonium remains in the germinal epithelium near the basement membrane to repeat process later on. The active spermatogonium will under go 4 mitotic divisions eventually forming 16 primary spermatocytes. Phase 2 (about 15 days duration) is mitotic division of primary spermatocytes during which the number of chromosomes is halved (meiosis-I). Phase 3 (generally a few hours) is division of secondary spermatocytes in to spermatids (meiosis Vii). Phase 4 takes place when the 4 spermatids are formed from each primary spermatocyte or 64 from each active spermatogonium. Spermatocytogenesis is followed by a stage where the spermatids become fully mature to form spermatozoans.

### Genes for Modification

There are a variety of biological molecules expressed on or in a sperm Mammalian sperm proteins are, in general, well conserved so that proteins on sperm of one mammal have counterparts in other species. Artisans are accustomed to locating the counterparts to proteins across species once a protein in a particular species has been identified. One category is the sperm fibrous sheath proteins. Chriva-Internati et al., Cancer Immunity, 2008, (8):8-13, reviews this group. The flagellum of spermatozoa has: (a) the connecting piece; (b) the middle piece that includes mitochondria; (c) the principal piece, and (d) the short end piece. The major cytoskeletal structures are the axoneme, the outer dense fibers, and the fibrous sheath (FS). The FS underlies the plasma membrane, and surrounds the axoneme and outer dense fibers. The FS seems to serve as a scaffold for glycolytic enzymes and signaling molecules. Several proteins localized in the FS have been identified, including, for instance, Sp17, CABYR, AKAP3, AKAP4, TAKAP-80, Rhopilin, Ropporin, GSTM5 and fibrousheathin.

Glutathione S-transferases (GSTs) localized at the sperm surface, see Hemachand et al., J. Cell Science, 2002, 115(10):2053-2065. GSTs are a family of enzymes that catalyze a number of glutathione (GSH)-dependent reactions and have been primarily described as cytosolic or microsomal detoxification enzymes that are also capable of functioning as intracellular binding proteins.

Naaby-Hansen reviewed sperm proteins in a dissertation “Functional and immunological analysis of the human sperm proteome”, Dan Med J, 2012, 59(3):B4414. About 200 sperm surface proteins were identified with labeling techniques. These proteins can be isolated and used to identify their corresponding gene. In fact, some of these were actually isolated and microsequenced, and two of the microsequences were used to make primers for cloning two sperm surface proteins designated SAMP14 and SAMP32. Another sperm surface protein is PH-20 (localized over the entire surface of sperm from several mammalian species). CD52 is an epitope on the protein SAGA-1 and is also the antigen for sperm-binding monoclonal antibody S19; SAGA-1 has been localized over all the surface domains in human sperm. The protein identified as serum amyloid P-component (SAP) is an abundant calcium-binding surface protein with MW of 26.5 kDa. 80K-H is another sperm surface protein; it is a multifunctional Ca2+-sensor with various functions including PKC pathways. Further, the calcium binding opsonin SAP and the three calcium-binding HSP70 chaperones HYOU1, HSPA5 and HSPA2 are known constituents of sperm plasma membrane.

Naaby-Hansen and Herr, J. Reprod. Immunol., 2010, 84(1):32-40 used mass spectrometry and Edman degradation to elucidate the identities of sperm proteins isolated from sperm surfaces labeled with biotin and radioiodine. They reported that seven members from four different heat shock protein (HSP) families were identified including HYOU1 (ORP150), FISPC1 (FISP86), HSPA5 (Bip), HSPD1 (HSP60), and several isoforms of the two testis-specific HSP70 chaperones HSPA2 and HSPAlL. An antiserum raised against the testis specific HSPA2 chaperone reacted with three 65 kDa HSPA2 isoforms and three high molecular weight surface proteins (78-79 kDa, 84 kDa and 90-93 kDa). These proteins, together with seven 65 kDa HSP70 forms, reacted with human anti-sperm IgG antibodies that blocked in vitro fertilization in humans Three of these surface biotinylated human sperm antigens were immunoprecipitated with a rabbit antiserum raised against a linear peptide epitope in Chlamydia trachomatis HSP70. The results indicated that diverse HSP chaperones are accessible for surface labeling on human sperm.

Fàbrega et al., Reproductive Biology and Endocrinology, 2011, 9(96):1-13 noted that spermatozoa surface proteins include fertilin, an heterodimer complex composed of two integral membrane glycoproteins named alpha-fertilin (ADAM-1) and beta-fertilin (ADAM-2) (PH-30 alpha and PH-30 beta in guinea pig), and several other ADAMs have been reported to be involved in sperm-oocyte recognition and in membrane fusion. ADAM stands for “A Disintegrin And Metalloprotease”, a family with defined features including a pro-domain, a metalloprotease, a disintegrin and a cysteine-rich domain, EGF-like repeats, a transmembrane domain and a carboxy-terminal cytosolic tail. Fàbrega et al. studied ADAMs in a boar model.

Larson and Miller, Biol Reprod., 1997, 57:442-453. Sperm from a variety of mammalian species express beta 1, 4 Galactosyltransferase (GalTase) on their surface. The authors performed GalTase enzyme assays on sperm from six species, and all six expressed GalTase on their surface. The amounts of GalTase varied between species. Guinea pig, mouse, and rat sperm had higher levels of GalTase than bovine, porcine, and rabbit sperm.

Dorus et al., Mol. Biol. Evol., 2010, 27(6):1235-1246 surveyed sperm proteins and reported a variety on the membrane or elsewhere in the sperm. Sperm proteins and genes localized to the cell membrane or to the whole sperm were identified and distinguished from each other. Many proteins in or on a sperm are known. Dorus provides a lengthy description and list of many proteins, the location, and the genes that express the same.

Genes in one livestock species consistently have orthologs in other livestock species, as well as in humans and mice. Humans and mice genes consistently have orthologs in livestock, particularly among cows, pigs, sheep, goats, chicken, and rabbits. Genetic orthologs between these species and fish is often consistent, depending upon the gene's function. Biologists are familiar with processes for finding gene orthologs so genes may be described herein in terms of one of the species without listing orthologs. Embodiments describing the inactivation, labeling, or epitope tagging of a gene thus include inactivation, labeling, or epitope tagging of orthologs that have the same or different names in other species. There are general genetic databases as well as databases that are specialized to identification of genetic orthologs.

Artisans may prepare fusion proteins using techniques known in these arts. Embodiments include a vector for, and methods of, transfecting a cell to thereby engineer the cell to make the fusion protein in vivo, with the cell being transfected in vitro, ex vivo, or in vivo, and with the cell being a member of a tissue implant or distinct therefrom. The following U.S. patent applications are hereby incorporated by reference herein for all purposes, including the purposes of making fusion proteins, with the instant specification controlling in case of conflict: 5227293, 5358857, 5885808, 5948639, 5994104, 6512103, 6562347, 6905688, 7175988, 7704943, US 2002/0004037, US 2005/0053579, US 2005/0203022, US 2005/0250936, US 2009/0324538.

### Sorting

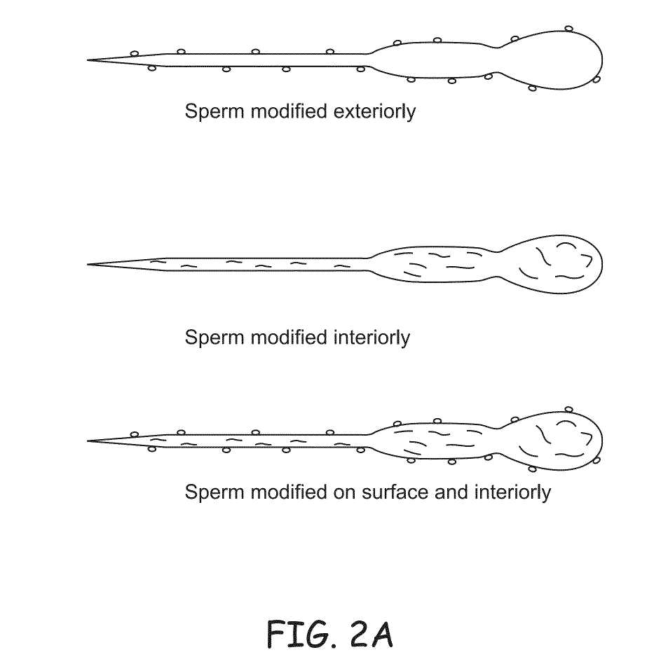

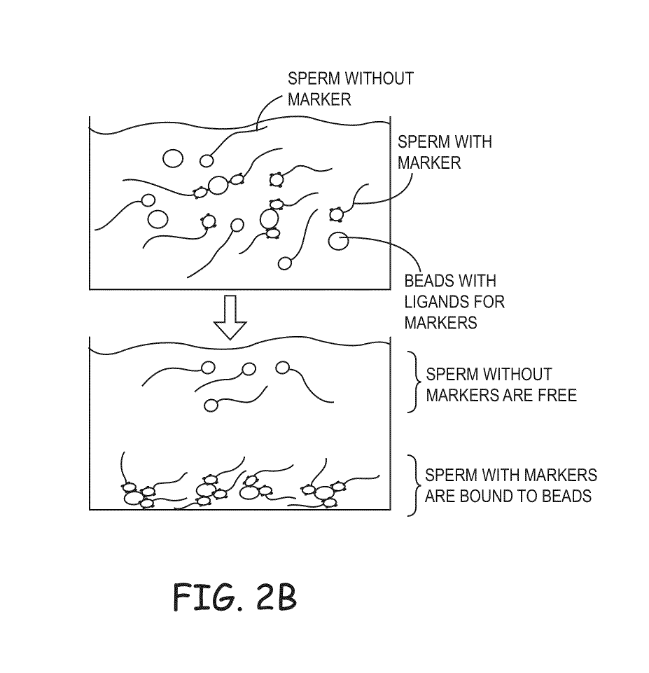

FIG. 2A depicts embodiments of genetically modified sperm: sperm modified on the surface; sperm modified interiorly; sperm modified both interiorly and on the surface. Interior modifications are useful for sorting processes based on visualization techniques or other selection processes based on, e.g., survival or other changes, such as altered motility. The markers can be chosen so that they are bound, or specifically bound, by ligands. FIG. 2B depicts sperm expressing a marker binding to beads that comprise a ligand that binds the markers. The sperm are exposed to the beads, marked sperm bind the beads via the ligands, and the beads are separated from the unbound sperm, e g., by magnetism, centrifugation, or gravity. Alternatives include, for example, immobilizing the ligands to beads in a column that receives the sample, placing a dipstick coated with the ligands into the sample, and coating a beaker or container walls with the ligand. Another alternative is to bind a plurality of ligands to a soluble material and allow the material to cross-link marked sperm to create a physical-phase separation, or a precipitation.

Other sorting methods rely on visualization, which is a broad term for generating an image. The visualization may be in the wavelength of visible light, fluorescent, or rely on radiopacity. Tools such as flow cytometry may be used. Markers may be on or inside a sperm cell for visualization.

Other sorting members rely on an electrostatic charge. Flow cytometry devices that separate cells by charge are known. The markers can be electrostatically active to produce a surface charge for sorting the cells with the marker from cells that do not express the marker.

Another embodiment of sorting relies on killing sperm cells by binding a toxic factor to a marker. The toxic factor causes cell death or marks the cell for destruction by a poison or other biological means, e.g., natural killer cells. Examples of a toxic factor are poisons and apoptotic factors. For instance, a fusion of enterotoxins may be made: enterotoxins have various modes of action. For instance, diphtheria toxin causes the formation of a hole, or pore, in the host cell membrane. Another example of a pore-forming exotoxin is the aerolysin produced by Aeromonas hydrophila. A second type of enterotoxin is superantigen toxin. Superantigen toxins work by stimulating T-cells. Examples of superantigen exotoxins include that from Staphylococcus aureus Streptococcus pyogenes. Protocols may include in vitro preparations of immune cells that are responsive to kill cells having the ligand-toxin fusion molecule. A complement-mediated lysis system, for instance, can be used to disable sperm that express the marker, e.g., see (Hauschild et al., 2011) wherein bi-allelic null cells could be enriched by FACS for the absence of a GGTA1-dependent surface epitope. A third type of enterotoxin is A-B toxin. An A-B toxin consists of two or more toxin subunits that work together. Typically the A subunit binds to the host cell wall and forms a channel through the membrane. The charmel allows the B subunit to get into the cell. An example of an A-B toxin is the enterotoxin produced by Vibrio cholerae. A ligand that is bound to the toxic factor is mixed with sperm carrying a marker. The marker binds the ligand so that the toxic factor is brought into proximity with the cell.

Other embodiments provide for a toxic factor to be expressed inside the sperm cell. The cell dies as a result. Therefore gametes with a Y chromosome that expresses a toxic factor will die, leaving sperm with an X chromosome. This embodiment is described in detail in U.S. Ser. No. 61/870,558 filed Aug. 27, 2013 and U.S. Ser. No. 61/829,656 filed May 31, 2013, and also copending U.S. Ser. No. ______ entitled GENETICALLY STERILE ANIMALS filed at about the same date as the present application, each of which is hereby incorporated by reference herein for all purposes.

Another embodiment relates to the expression of an antidote to a poison. The sperm are placed into a solution that contains the poison. Sperm that lack the antidote are destroyed.

Positive and/or negative selection may be used. The marker may therefore be used to identify sperm cells that are to be used, or those that are not desired for use. In some cases, the gender with the marker may be preferred while, at other times, the gender without the marker may be preferred. If two different markers are used on different sex chromosomes, then one marker may be positively selected while another marker is negatively selected. A sex chromosome may express one, or more than 1 marker.

### Selective Binding Moieties

A binding moiety that selectively binds a marker may be a ligand or a chemical group that binds by a more general interaction such as electrostatic, ionic, or hydrophobic affinity. A ligand is a chemical moiety that exhibits specific binding to its target. Ligand interactions include enzyme-substrate, receptor-to-ligand, and antibody-antigen binding events.

Antibodies may readily be generated for a protein. A marker that is expressed at a surface of a sperm is very likely to be specifically bound by an antibody that can easily be generated experimentally. Methods for using a portion of an antibody that has binding affinity for its target are well known. The term antigen, in this context, refers to a site recognized by a host immune system that responds to the antigen. Antigen selection is known in the arts of raising antibodies, among other arts. The term antibody fragment refers to a portion of an antibody that retains the antigen-binding function of the antibody. The fragment may literally be made from a portion of a larger antibody or alternatively may be synthesized de novo. Antibody fragments include, for example, a single chain variable fragment (scFv) An scFv is a fusion protein of the variable regions of the heavy (VH) and light chains (VL) of immunoglobulin, connected with a linker peptide, e.g., about 10 to about 50 amino acids. The linker can either connect the N-terminus of the VH with the C-terminus of the VL, or vice versa. The term scFv includes divalent scFvs, diabodies, triabodies, tetrabodies and other combinations of antibody fragments. Antibodies have an antigen-binding portion referred to as the paratope. The term peptide ligand refers to a peptide that is not part of a paratope.

Aptamers can generally be made to specifically bind markers with high affinity. DNA and RNA aptamers may be used to provide non-covalent binding. As they are only composed of nucleotides, aptamers are promising biomolecular targeting moieties in that screening methodologies are well established, they are readily chemically synthesized, and pose limited side-effect toxicity and/or immunogenicity due to their rapid clearance in vivo (Keefe, Pai, et al., 2010 Aptamers are oligonucleic acids or peptides that bind to a specific target molecule. Aptamers are usually created to bind a target of interest by selecting them from a large random sequence pool. Aptamers can be classified as DNA aptamers, RNA aptamers, or peptide aptamers. Nucleic acid aptamers are nucleic acid species that have been engineered through repeated rounds of in vitro selection or Systematic Evolution of Ligands by Exponential Enrichment (SELEX) method (Archemix, Cambridge, Mass., USA) (Sampson, 2003) to specifically bind to targets such as small molecules, proteins, nucleic acids, cells, tissues and organisms. Peptide aptamers typically have a short variable peptide domain, attached at both ends to a protein scaffold. Peptide aptamers are proteins that are designed to interfere with other protein interactions inside cells. They consist of a variable peptide loop attached at both ends to a protein scaffold. This double structural constraint greatly increases the binding affinity of the peptide aptamer to be comparable to an antibody. The variable loop length is typically composed of about ten to about twenty amino acids, and the scaffold is a protein which has good solubility and is compact. For example the bacterial protein Thioredoxin-A is a scaffold protein, with the variable loop being inserted within the reducing active site, which is a -Cys-Gly-Pro-Cys- loop in the wild protein, the two Cysteines lateral chains being able to form a disulfide bridge. Some techniques for making aptamers are detailed in Lu et al., Chem. Rev., 2009, 109(5):1948-1998, and also in U.S. Pat. No. 7,892,734, U.S. Pat. No. 7,811,809, US 2010/0129820, US 2009/0149656, US 2006/0127929, and US 2007/0111222.

Peptide sequences can be generated to specifically bind markers. Several methods exist for affinity selection of binding proteins or polypeptides such as phage display, yeast surface display, mRNA display or peptide-on-bead display. See: Boder E T, Wittrup K D. Smith G P, Petrenko V A. Phage Display. Chem. Rev., 1997, 97-2:391-410; Yeast surface display for screening combinatorial polypeptide libraries. Nat. Biotechnol., 1997, 15-6:553-557; Xu L, Aha P, Gu K, Kuimelis R G, Kurz M, Lam T, Lim A C, Liu H, Lohse P A, Sun L, Weng S, Wagner R W, Lipovsek D. Directed evolution of high-affinity antibody mimics using mRNA display. Chem. Biol., 2002, 9-8:933-942; Lam K S, Lebl M, Krchnak V. The “One-Bead-One-Compound” Combinatorial Library Method. Chem. Rev., 1997, 97-2:411-448 Zacher A N, 3rd, Stock C A, Golden J W, 2nd, Smith G P. A new filamentous phage cloning vector: fd-tet. Gene, 1980, 9-1-2:127-140.

In all of these cases, there is a high expectation of success that a protein can be expressed in a sperm and a specifically binding element can be produced to bind it, so long as the protein is accessible at the surface of the sperm.

### Genetic Modifications of Animals

An embodiment of a genetically modified animal, the animal comprising cells that comprise a chromosome that comprises an exogenous gene under control of a promoter selectively activated in gametogenesis. An animal may be created by genetic modification of a cell or embryo. One or both of the sex chromosomes are modified, i.e., the X- or the Y-chromosome. The marker that is expressed by the exogenous gene is under control of an expression element that is selective for a stage of spermatogenesis. The expression element is, e.g., a promoter, a micro RNA

Cells developing into sperm share cytoplasm deep into the spermatogenic cycle. Cytoplasm sharing comes to an end when cytoplasmic bridges between sister-cells drop away. Genetic modifications that express a gene before this sharing is ended will not be effective because some of the sisters sharing cytoplasm have an X chromosome and some have a Y chromosome. One group of embodiments set forth herein uses genetic expression elements (promoters and/or microRNAs) to control expression. Another group of embodiments places the marker on a protein that is expressed after the cytoplasmic bridges are gone, e.g., certain tail proteins as set forth herein.

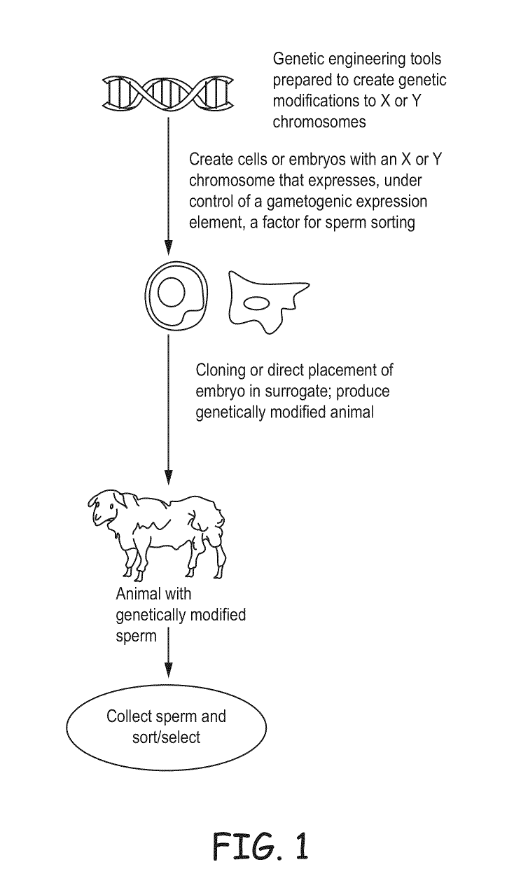

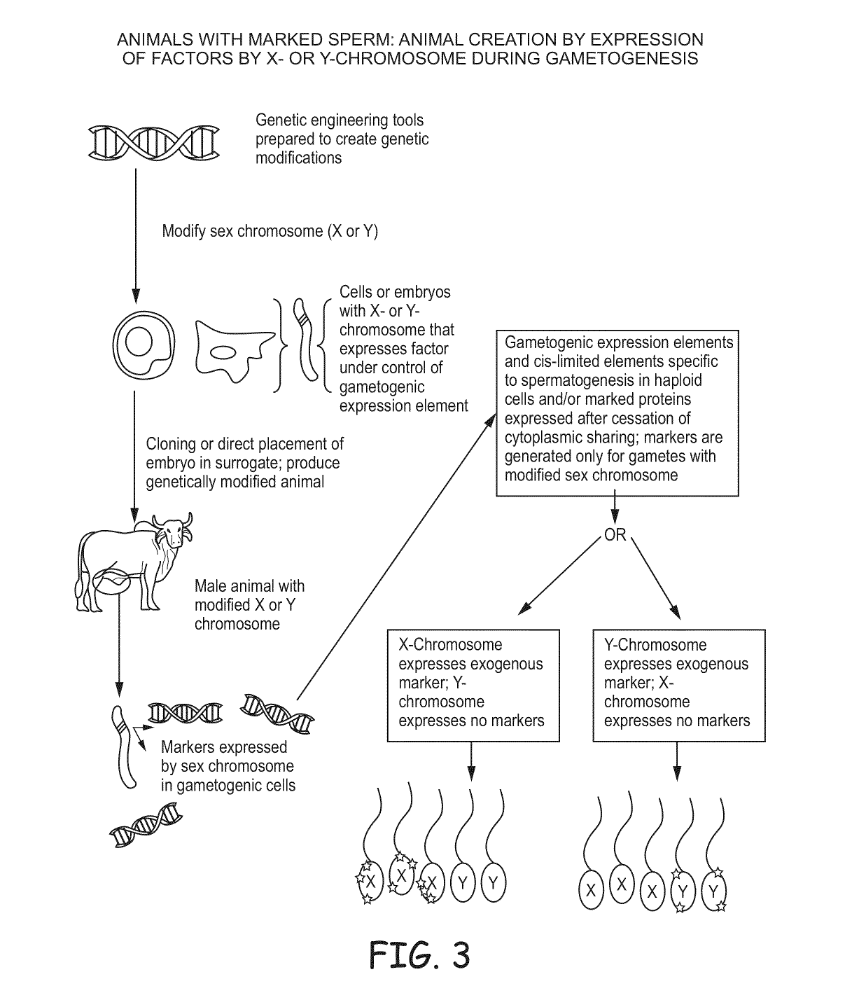

FIGS. 1 and 3 depict a process of making a genetic modification of an animal. Cells are modified and used to make embryos, e.g., with cloning techniques or embryos are directly treated to modify the cells. The cells are modified with a factor under control of a gametogenic expression element. The factor could be an exogenous gene on an X, Y, or autosome, or it could be an element that regulates/disrupts a gene. As gametogenesis proceeds, the factor is activated. The factor can have various effects, for example: killing or expressing a marker in gametes with an X chromosome, killing or expressing a marker in gametes with a Y chromosome, with the marker being as described herein, e.g., visualization, positive selection, negative selection, co-selection, survival, ligand, antigen.

Animals may be made that are mono-allelic or bi-allelic for a chromosomal modification, using methods that either leave a marker in place, allow for it to be bred out of an animal, or by methods that do not place a marker in the animal. For instance, the inventors have used methods of homologous dependent recombination (HDR) to make changes to, or insertion of exogenous genes into, chromosomes of animals. Tools such as TALENs and recombinase fusion proteins, as well as conventional methods, are discussed elsewhere herein. Some of the experimental data supporting genetic modifications disclosed herein is summarized as follows. The inventors have previously demonstrated exceptional cloning efficiency when cloning from polygenic populations of modified cells, and advocated for this approach to avoid variation in cloning efficiency by somatic cell nuclear transfer (SCNT) for isolated colonies (Carlson et al., 2011). Additionally, however, targeting endonuclease, e.g., TALEN-mediated genome modification, as well as modification by recombinase fusion molecules, provides for a bi-allelic alteration to be accomplished in a single generation. For example, an animal homozygous for a knocked-out gene may be made by SCNT and without inbreeding to produce homozygosity. Gestation length and maturation to reproduction age for livestock such as pigs and cattle is a significant barrier to research and to production. For example, generation of a homozygous knockout from heterozygous mutant cells (both sexes) by cloning and breeding would require 16 and 30 months for pigs and cattle respectively. Some have reduced this burden with sequential cycles of genetic modification and SCNT (Kuroiwa et al., 2004) however, this is both technically challenging and cost prohibitive. The ability to routinely generate bi-allelic KO cells prior to SCNT is a significant advancement in large animal genetic engineering. Bi-allelic knockout has been achieved in immortal cells lines using other processes such as ZFN and dilution cloning (Liu et al., 2010). Another group recently demonstrated bi-allelic KO of porcine GGTA1 using commercial ZFN reagents (Hauschild et al., 2011) where bi-allelic null cells could be enriched by FACS for the absence of a GGTA1-dependent surface epitope. While these studies demonstrate certain useful concepts, they do not show that animals or livestock could be modified because simple clonal dilution has generally not been feasible for primary fibroblast isolates (fibroblasts grow poorly at low density) and biological enrichment for null cells is not available for the majority of genes.

The inventors have previously shown that transgenic primary fibroblasts can be effectively expanded and isolated as colonies when plated with non-transgenic fibroblasts at densities greater than 150 cells/cm² and subjected to drug selection using a transposon co-selection technique (Carlson et al., 2011, U.S. Pub. No. 2011/0197290). It was further shown (see U.S. Ser. No. 13/404,662 filed Feb. 24, 2012) that puromycin resistant colonies were isolated for cells treated with six TALEN pairs and evaluated their genotypes by SURVEYOR assay or direct sequencing of PCR products spanning the target site. In general, the proportion of indel positive clones was similar to predictions made based on day 3 modification levels. Bi-allelic KO clones were identified for 5 of 6 TALEN pairs, occurring in up to 35% of indel positive cells. Notably, the frequency of bi-allelic KO clones for the majority of TALEN pairs exceeds what would be predicted if the cleavage of each chromosome is treated as an independent event.

TALEN-induced homologous recombination eliminates the need for linked selection markers. TALENs may be used to precisely transfer specific alleles into a livestock genome by homology dependent repair (HDR). In a pilot study, a specific 11 bp deletion (the Belgian Blue allele) (Grobet et al., 1997; Kambadur et al., 1997) was introduced into the bovine GDF8 locus (see U.S. Ser. No. 13/404,662 filed Feb. 24, 2012). When transfected alone, the btGDF8.1 TALEN pair cleaved up to 16% of chromosomes at the target locus. Co-transfection with a supercoiled homologous DNA repair template harboring the 11 bp deletion resulted in a gene conversion frequency (HDR) of up to 5% at day 3 without selection for the desired event. Gene conversion was identified in 1.4% of isolated colonies that were screened. These results demonstrated that TALENs can be used to effectively induce HDR without the aid of a linked selection marker. Example 1 provides experimental data showing that a Y-chromosome, or other chromosomes, may be genetically altered by using, for instance, TALENs. TALENs are discussed in more detail elsewhere herein.

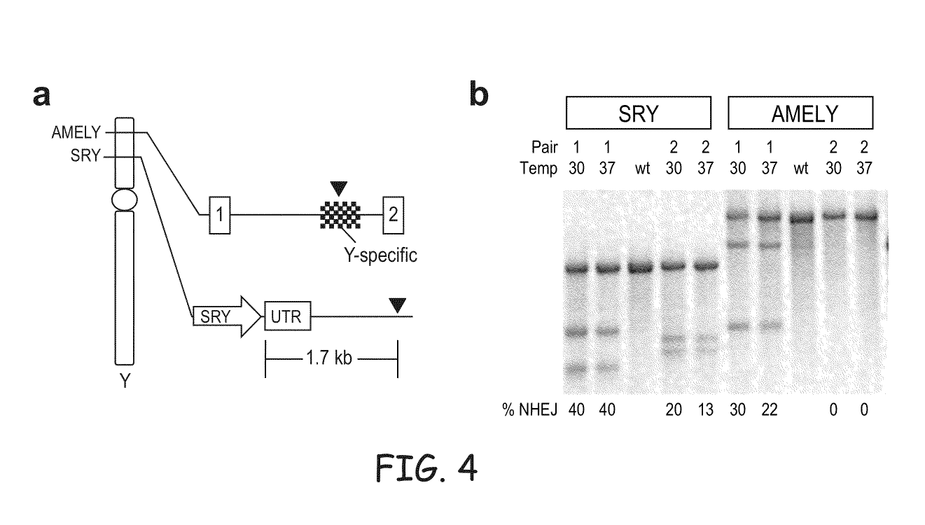

Example 1, see FIG. 4, describes TALENs directed to targets at the Y chromosome. Three TALENs pairs showed activity. Accordingly, cells can be made with indels on the Y chromosome, and animals from the cells. Example 2 provides methods for a TALEN-mediated genome modification to achieve a bi-allelic knockout in single generation. Gestation length and maturation to reproduction age for pigs and cattle is significant; for example, generation of a homozygous knockout from heterozygous mutant cells (both sexes) by cloning and breeding would require 16 and 30 months for pigs and cattle respectively. Bi-allelic knockout has been achieved in immortal cells lines using ZFN and dilution cloning. (Liu et al., 2010) Another group recently demonstrated bi-allelic knockout of porcine GGTA1 using commercial ZFN reagents (Hauschild et al., 2011) where bi-allelic null cells could be enriched by FACS for the absence of a GGTA1-dependent surface epitope. While these other studies are useful, they use simple clonal dilution. Such processes are not feasible for the majority of primary fibroblast isolates and biological enrichment for null cells is not available for the majority of genes. In Example 2, however, primary cells were used, based on a method that permits expansion of individual colonies to screen for bi-allelic knockout. Example 3 demonstrates an alternative method of modifying cells useful for making cloned animals. Examples 4 demonstrates other methods of making cells for cloning, specifically, methods involving single-stranded oligonucleotides as HDR templates. Example 5 uses the single-stranded oligonucleotide processes to move genes from one species to another in an efficient process that is free of markers.

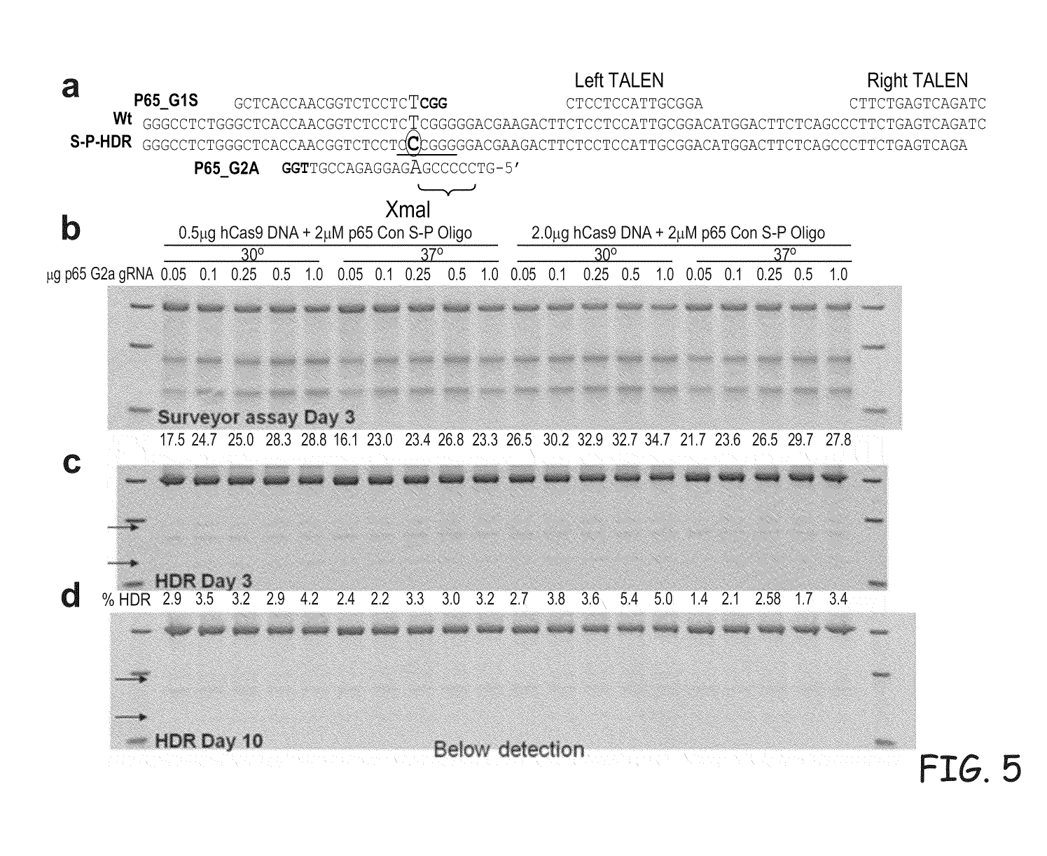

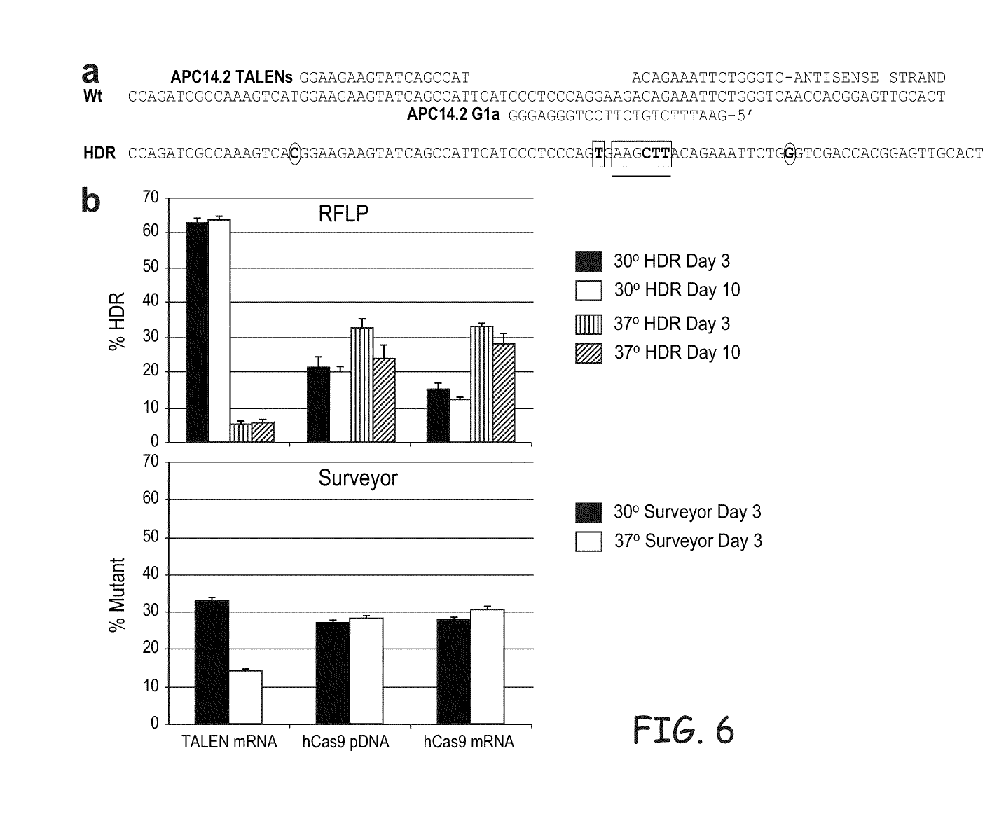

Examples 6-8 describe Cas9/CRISPR nuclease editing of genes. Examples 7 and 8 are Cas9/CRISPR results, showing efficient production of double stranded breaks at the intended site. Such breaks provide opportunities for gene editing by HDR template repair processes. CRISPR/Cas9-mediated HDR was lower than 6 percent at day-3 and below detection at day-10 (FIG. 5). Analysis of CRISPR/Cas9 induced targeting at a second locus, ssAPC14.2, was much more efficient, but still did not reach the level of HDR induced by TALENs at this site, about 30% versus 60% (FIG. 6). Cas9/CRISPR was an effective tool, as shown by these experiments.

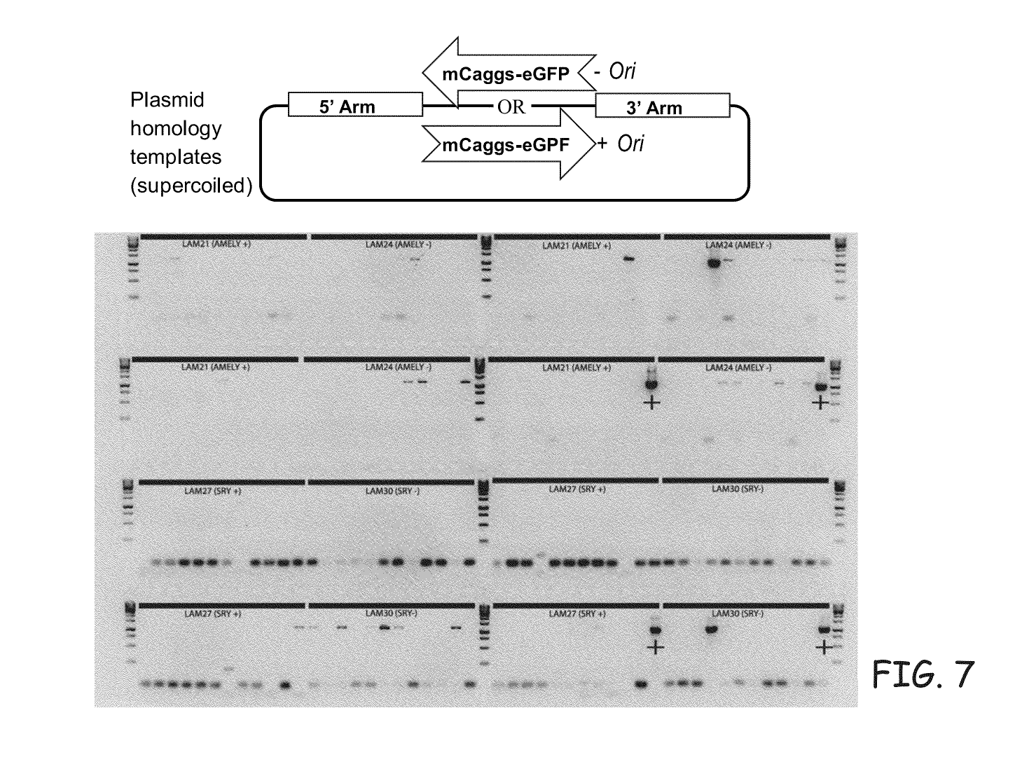

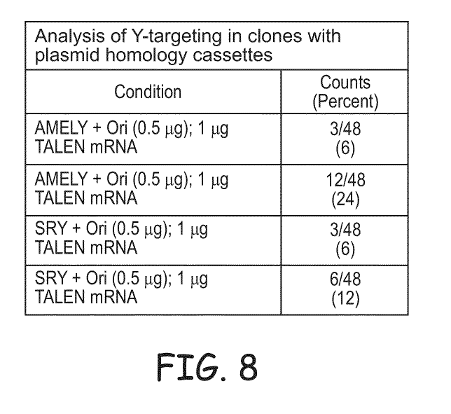

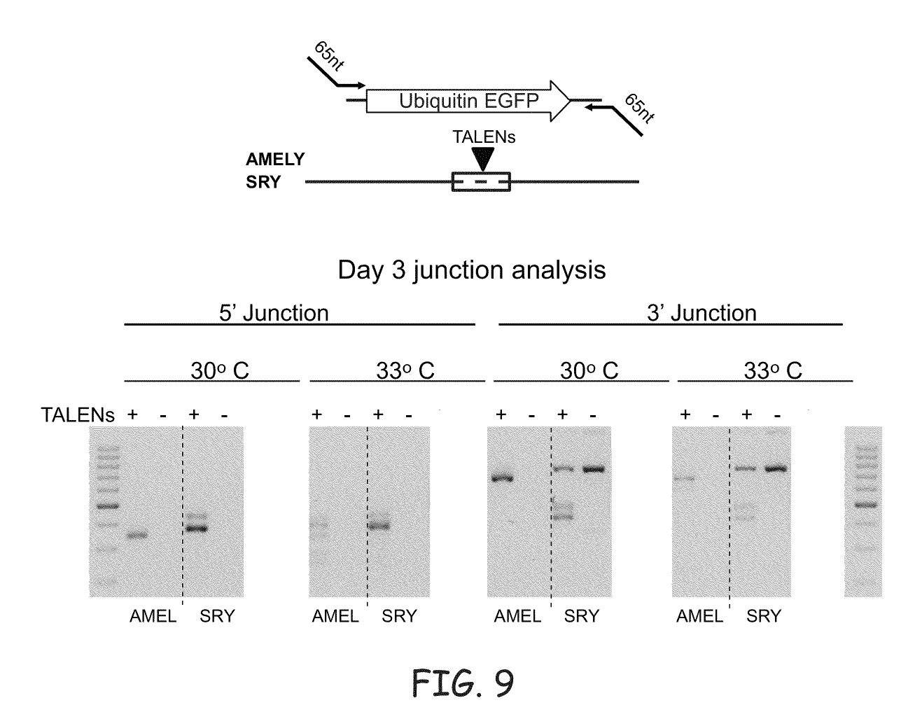

Examples 9 and 10 describe targeting of the Y-chromosome with either a plasmid cassette (FIGS. 7 and 8) or with a linear short homology template (FIGS. 9-11). Both techniques used TALENs to create a double strand break at the intended targeting site and homology templates directed the gene of interest to the target location. The efficiency was between 1 and 24% with both methods being effective.

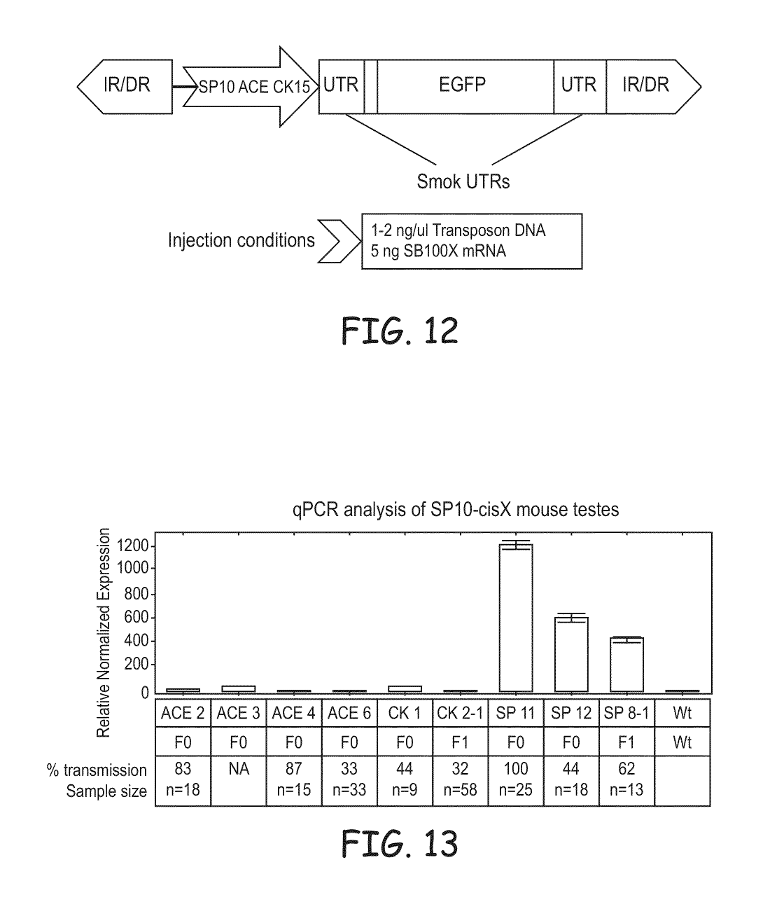

Example 11, see FIGS. 12-15 describes a series of vectors created to carry a presumptive cis-restricted transgene under the direction of either the porcine ACE, CK-15 or SP10 promoters, all originally cloned by Applicants' team based on comparative data with mice. Consistent with the results of the qPCR, signal was detected in only sperm from SP10 founders. Embodiments of the invention include one or more cis-restricted transgene in a gamete or involved in gametogenesis under the direction of a promoter, e.g., tissue-specific promoter.

Embodiments of the invention include a method of making a genetically modified animal, said method comprising exposing embryos or cells to a vector or an mRNA encoding a targeting nuclease (e.g., meganuclease, zinc finger, TALENs), with the targeting nuclease specifically binding to a target chromosomal site in the embryos or cells to create a change to a cellular chromosome, cloning the cells in a surrogate mother or implanting the embryos in a surrogate mother, with the surrogate mother thereby gestating an animal that is genetically modified without a reporter gene and only at the targeted chromosomal site. The targeted site may be one as set forth herein, e.g., the various genes described herein. Targeted nuclease systems include a motif that binds to the cognate DNA, either by protein-to-DNA binding, or by nucleic acid-to-DNA binding. The efficiencies reported herein are significant. The inventors have disclosed further techniques elsewhere that further increase these efficiencies.

### Gametogenesis and Gametogenic Expression Elements

Gametogenesis refers to the biological process by which germ line precursor cells undergo cell division and differentiation to form mature haploid gametes. Animals produce gametes through meiosis in the gonads. Primordial germ cells (PGCs) form gametogonia during development. Female gametognia undergo oogenesis, which has sub-processes of oocytogenesis, ootidogenesis, and maturation to form an ovum (sometimes referred to as oogenesis). Male gametognia undergo spermatogenesis. The gametogonia are precursors to male primary sperm cells (diploid) that undergo meiosis to produce spermatogonial (diploid) that give rise to primary spermatocytes (diploid). Primary spermatocytes undergo meiosis to form secondary spermatocytes (haploid) that form spermatids (haploid) that develop into mature spermatozoa (haploid), also known as sperm cells. The seminiferous tubules of the testes are the starting point for the process, where stem cells adjacent to the inner tubule wall divide in a centripetal direction beginning at the walls and proceeding into the innermost part to produce spermatids. Maturation of the spermatids occurs in the epididymis. Research in mice or rats has shown that seminiferous tubules of a first animal can receive tissue and/or spermatogonial cells from a donor animal so that the donated cells mature into spermatozoa that are functional. The recipient seminiferous tubules can effectively host the spermatogenic processes for donor cells.

Gametogenic promoters are promoters that are selective for gametogenic processes. Some gametogenic promoters act before the meiotic stages of gametogenesis while others are specifically activated at various points in the process of gametogenesis. Of particular interest are promoters active in spermatogenesis only after cytoplasmic sharing has stopped. Embodiments include an exogenous gene placed into a cell or embryo under control of a promoter selective for gametogenesis or selectively activated during one or more gametogenic subprocesses chosen from the group consisting of oocytogenesis, ootidogenesis, oocyte maturation, spermatogenesis, maturation into spermatogonial cells, maturation into primary spermatocytes, maturation into secondary spermatocytes, maturation into spermatids, and maturation into sperm cells. Some promoters are generally active during gametogenesis while others are activated beginning at a certain subprocess but may continue through other phases of gametogenesis. Embodiments further include an exogenous gene placed into a cell or embryo under control of a tissue-specific promoter selective for gametogenic processes: for example, a tissue specific promoter selectively active in a tissue selected from the group consisting of testes, seminiferous tubules, and epididymis. A pre and post meiotic gametogenesis promoter is the cyclin A1 promoter, which is active not only in pachytene spermatocytes but also in earlier phases of spermatogenesis (Müller-Tidow et al., Int J Mol Med., 2003 March, 11(3):311-315; Successive increases in human cyclin A1 promoter activity during spermatogenesis in transgenic mice). The promoter of SP-10 (−408/+28 or the −266/+28; referred to as SP-10 promoters) is directed only to spermatid-specific transcription. In fact, in transgenic mice, despite transgene integration adjacent to the pan-active CMV enhancer, the −408/+28 promoter maintained spermatid-specificity and no ectopic expression of the transgene resulted (P Reddi, et al. Spermatid-specific promoter of the SP-10 gene functions as an insulator in somatic cells. Developmental Biology (2003) Volume: 262, Issue: 1, Pages: 173-182). The 400-bp regulatory region of the stimulated by retinoic acid gene 8 (Stra8) promoter (referred to as the Stra8 promoter) is selectively active in meiotic and postmeiotic germ cells and not in undifferentiated germ cells (Antonangeli et al., Expression profile of a 400-bp Stra8 promoter region during spermatogenesis; Microscopy Research and Technique (2009) Volume: 72, Issue: 11, Pages: 816-822).

The inventors have developed precise, high frequency editing of a variety of genes in about various livestock cells and/or animals that are useful for agriculture, for research tools, or for biomedical purposes. These livestock gene-editing processes include TALEN and CRISPR/Cas9 stimulated homology-directed repair (HDR) using, e.g., plasmid, rAAV and oligonucleotide templates. These processes have been developed by the inventors to achieve efficiencies that are so high that genetic changes can be made without reporters and/or without selection markers. Moreover, the processes can be used in the founder generation to make genetically modified animals that have only the intended change at the intended site. For instance, processes and data for targeting nucleases are provided in U.S. Ser. No. 14/154,906 filed Jan. 14, 2014, which is hereby incorporated herein by reference. See Table 1: Frequencies for recovery of colonies with HDR alleles.

### Homology Directed Repair (HDR)

Homology directed repair (HDR) is a mechanism in cells to repair ssDNA and double stranded DNA (dsDNA) lesions. This repair mechanism can be used by the cell when there is an HDR template present that has a sequence with significant homology to the lesion site. Specific binding, as that term is commonly used in the biological arts, refers to a molecule that binds to a target with a relatively high affinity compared to non-target tissues, and generally involves a plurality of non-covalent interactions, such as electrostatic interactions, van der Waals interactions, hydrogen bonding, and the like. Specific hybridization is a form of specific binding between nucleic acids that have complementary sequences. Proteins can also specifically bind to DNA, for instance, in TALENs or CRISPR/Cas9 systems or by Gal4 motifs. Introgression of an allele refers to a process of copying an exogenous allele over an endogenous allele with a template-guided process. The endogenous allele might actually be excised and replaced by an exogenous nucleic acid allele in some situations but present theory is that the process is a copying mechanism. Since alleles are gene pairs, there is significant homology between them. The allele might be a gene that encodes a protein, or it could have other functions such as encoding a bioactive RNA chain or providing a site for receiving a regulatory protein or RNA.

The HDR template is a nucleic acid that comprises the allele that is being introgressed. The template may be a dsDNA or a single-stranded DNA (ssDNA). ssDNA templates are preferably from about 20 to about 5000 residues although other lengths can be used. Artisans will immediately appreciate that all ranges and values within the explicitly stated range are contemplated; e.g., from 500 to 1500 residues, from 20 to 100 residues, and so forth. The template may further comprise flanking sequences that provide homology to DNA adjacent to the endogenous allele or the DNA that is to be replaced. The template may also comprise a sequence that is bound to a targeted nuclease system, and is thus the cognate binding site for the system's DNA-binding member. The term cognate refers to two biomolecules that typically interact, for example, a receptor and its ligand. In the context of HDR processes, one of the biomolecules may be designed with a sequence to bind with an intended, i.e., cognate, DNA site or protein site.

### Site-Specific Nuclease Systems

Genome editing tools such as transcription activator-like effector nucleases (TALENs) and zinc finger nucleases (ZFNs) have impacted the fields of biotechnology, gene therapy and functional genomic studies in many organisms. More recently, RNA-guided endonucleases (RGENs) are directed to their target sites by a complementary RNA molecule. The Cas9/CRISPR system is a RGEN. tracrRNA is another such tool. These are examples of targeted nuclease systems: these systems have a DNA-binding member that localizes the nuclease to a target site. The site is then cut by the nuclease. TALENs and ZFNs have the nuclease fused to the DNA-binding member. Cas9/CRISPR are cognates that find each other on the target DNA. The DNA-binding member has a cognate sequence in the chromosomal DNA. The DNA-binding member is typically designed in light of the intended cognate sequence so as to obtain a nucleolytic action at or near an intended site. Certain embodiments are applicable to all such systems without limitation; including, embodiments that minimize nuclease re-cleavage, embodiments for making SNPs with precision at an intended residue, and placement of the allele that is being introgressed at the DNA-binding site.

### TALENs

The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA or a TALEN-pair.

The cipher for TALs has been reported (PCT Application WO 2011/072246) wherein each DNA binding repeat is responsible for recognizing one base pair in the target DNA sequence. The residues may be assembled to target a DNA sequence. In brief, a target site for binding of a TALEN is determined and a fusion molecule comprising a nuclease and a series of RVDs that recognize the target site is created. Upon binding, the nuclease cleaves the DNA so that cellular repair machinery can operate to make a genetic modification at the cut ends. The term TALEN means a protein comprising a Transcription Activator-like (TAL) effector binding domain and a nuclease domain and includes monomeric TALENs that are functional per se as well as others that require dimerization with another monomeric TALEN. The dimerization can result in a homodimeric TALEN when both monomeric TALEN are identical or can result in a heterodimeric TALEN when monomeric TALEN are different. TALENs have been shown to induce gene modification in immortalized human cells by means of the two major eukaryotic DNA repair pathways, non-homologous end joining (NHEJ) and homology directed repair. TALENs are often used in pairs but monomeric TALENs are known. Cells for treatment by TALENs (and other genetic tools) include a cultured cell, an immortalized cell, a primary cell, a primary somatic cell, a zygote, a germ cell, a primordial germ cell, a blastocyst, or a stem cell. In some embodiments, a TAL effector can be used to target other protein domains (e.g., non-nuclease protein domains) to specific nucleotide sequences. For example, a TAL effector can be linked to a protein domain from, without limitation, a DNA 20 interacting enzyme (e.g., a methylase, a topoisomerase, an integrase, a transposase, or a ligase), a transcription activators or repressor, or a protein that interacts with or modifies other proteins such as histones. Applications of such TAL effector fusions include, for example, creating or modifying epigenetic regulatory elements, making site-specific insertions, deletions, or repairs in DNA, controlling gene expression, and modifying chromatin structure.

The term nuclease includes exonucleases and endonucleases. The term endonuclease refers to any wild-type or variant enzyme capable of catalyzing the hydrolysis (cleavage) of bonds between nucleic acids within a DNA or RNA molecule, preferably a DNA molecule. Non-limiting examples of endonucleases include type II restriction endonucleases such as FokI, HhaI, HindIa NocI, BbvCl, EcoRI, BglII, and AlwI. Endonucleases comprise also rare-cutting endonucleases when having typically a polynucleotide recognition site of about 12-45 basepairs (bp) in length, more preferably of 14-45 bp. Rare-cutting endonucleases induce DNA double-strand breaks (DSBs) at a defined locus. Rare-cutting endonucleases can for example be a homing endonuclease, a chimeric Zinc-Finger nuclease (ZFN) resulting from the fusion of engineered zinc-finger domains with the catalytic domain of a restriction enzyme such as FokI or a chemical endonuclease. In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences. Such chemical endonucleases are comprised in the term “endonuclease” according to the present invention. Examples of such endonuclease include I-See I, I-Chu L I-Cre I, I-Csin I, PI-See L PI-Tti L PI-Mitt I, I-Ceu I, I-See IL I- See III, HO, PI-Civ I, PI-Ctr L PI-Acte I, PI-Bsu I, PI-Dha I, PI-Dra L PI-Mav L PI-Meh L PI-Mil I, PI-Mga L PI-Mgo I, L PI-Mka L PI-Mle I, PI-Mma I, PI- 30 Msh L PI-Msm I, PI-Mth I, PI-IVItu I, PI-Mxe I, PI-Npu I, PI-Pfu L PI-Rma I, PI-Spb I, PI-Ssp L PI-Fae L PI-Mja I, PI-Pho L PI-Tag L PI-Thy I, PI-Tko I, PI-Tsp I, I-MsoI.

A genetic modification made by TALENs or other tools may be, for example, chosen from the list consisting of an insertion, a deletion, insertion of an exogenous nucleic acid fragment, and a substitution. The term insertion is used broadly to mean either literal insertion into the chromosome or use of the exogenous sequence as a template for repair. In general, a target DNA site is identified and a TALEN-pair is created that will specifically bind to the site. The TALEN is delivered to the cell or embryo, e.g., as a protein, mRNA or by a vector that encodes the TALEN. The TALEN cleaves the DNA to make a double-strand break that is then repaired, often resulting in the creation of an indel, or incorporating sequences or polymorphisms contained in an accompanying exogenous nucleic acid that is either inserted into the chromosome or serves as a template for repair of the break with a modified sequence. This template-driven repair is a useful process for changing a chromosome, and provides for effective changes to cellular chromosomes.

The term exogenous nucleic acid means a nucleic acid that is added to the cell or embryo, regardless of whether the nucleic acid is the same or distinct from nucleic acid sequences naturally in the cell. The term nucleic acid fragment is broad and includes a chromosome, expression cassette, gene, DNA, RNA, mRNA, or portion thereof. The cell or embryo may be, for instance, chosen from the group consisting of livestock, an artiodactyl, a cow, a swine, a sheep, a goat, a chicken, a rabbit, and a fish. The term livestock means domesticated animals that are raised as commodities for food or biological material. The term artiodactyl means a hoofed mammal of the order Artiodactyla, which includes cattle, deer, camels, hippopotamuses, sheep, and goats that have an even number of toes, usually two or sometimes four, on each foot.

Some embodiments involve a composition or a method of making a genetically modified livestock and/or artiodactyl comprising introducing a TALEN-pair into livestock and/or an artiodactyl cell or embryo that makes a genetic modification to DNA of the cell or embryo at a site that is specifically bound by the TALEN-pair, and producing the livestock animal/artiodactyl from the cell. Direct injection may be used for the cell or embryo, e.g., into a zygote, blastocyst, or embryo. Alternatively, the TALEN and/or other factors may be introduced into a cell using any of many known techniques for introduction of proteins, RNA, mRNA, DNA, or vectors. Genetically modified animals may be made from the embryos or cells according to known processes, e.g., implantation of the embryo into a gestational host, or various cloning methods. The phrase “a genetic modification to DNA of the cell at a site that is specifically bound by the TALEN”, or the like, means that the genetic modification is made at the site cut by the nuclease on the TALEN when the TALEN is specifically bound to its target site. The nuclease does not cut exactly where the TALEN-pair binds, but rather at a defined site between the two binding sites.

Some embodiments involve a composition or a treatment of a cell that is used for cloning the animal. The cell may be a livestock and/or artiodactyl cell, a cultured cell, a primary cell, a primary somatic cell, a zygote, a germ cell, a primordial germ cell, or a stem cell. For example, an embodiment is a composition or a method of creating a genetic modification comprising exposing a plurality of primary cells in a culture to TALEN proteins or a nucleic acid encoding a TALEN or TALENs. The TALENs may be introduced as proteins or as nucleic acid fragments, e.g., encoded by mRNA or a DNA sequence in a vector.

### Zinc Finger Nucleases

Zinc-finger nucleases (ZFNs) are artificial restriction enzymes generated by fusing a zinc finger DNA-binding domain to a DNA-cleavage domain. Zinc finger domains can be engineered to target desired DNA sequences and this enables zinc-finger nucleases to target unique sequences within complex genomes. By taking advantage of endogenous DNA repair machinery, these reagents can be used to alter the genomes of higher organisms. ZFNs may be used in method of inactivating genes.

A zinc finger DNA-binding domain has about 30 amino acids and folds into a stable structure. Each finger primarily binds to a triplet within the DNA substrate. Amino acid residues at key positions contribute to most of the sequence-specific interactions with the DNA site. These amino acids can be changed while maintaining the remaining amino acids to preserve the necessary structure. Binding to longer DNA sequences is achieved by linking several domains in tandem. Other functionalities like non-specific Fold cleavage domain (N), transcription activator domains (A), transcription repressor domains (R) and methylases (M) can be fused to a ZFPs to form ZFNs respectively, zinc finger transcription activators (ZFA), zinc finger transcription repressors (ZFR, and zinc finger methylases (ZFM). Materials and methods for using zinc fingers and zinc finger nucleases for making genetically modified animals are disclosed in, e.g., U.S. Pat. No. 8,106,255 US 2012/0192298, US 2011/0023159, and US 2011/0281306.

Vectors and Nucleic acids

A variety of nucleic acids may be introduced into cells, for knockout purposes, for inactivation of a gene, to obtain expression of a gene, or for other purposes. As used herein, the term nucleic acid includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained.

The target nucleic acid sequence can be operably linked to a regulatory region such as a promoter. Regulatory regions can be porcine regulatory regions or can be from other species. As used herein, operably linked refers to positioning of a regulatory region relative to a nucleic acid sequence in such a way as to permit or facilitate transcription of the target nucleic acid.

Any type of promoter can be operably linked to a target nucleic acid sequence. Gametogenic promoters or other expression elements are preferred in the case of making sperm with markers but a more general expression of markers can be effective. Types of promoters include, without limitation, tissue-specific promoters, constitutive promoters, inducible promoters, and promoters responsive or unresponsive to a particular stimulus. In other embodiments, a promoter that facilitates the expression of a nucleic acid molecule without significant tissue- or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter. See, for example, Xu et al. (2001) Hum. Gene Ther., 12:563; and Kiwaki et al. (1996) Hum. Gene Ther., 7:821.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban, et al., Proc. Natl. Acad. Sci. (1992) 89:6861, for a review of Cre/lox technology, and Brand and Dymecki, Dev. Cell (2004) 6:7. A transposon containing a Cre- or Flp-activatable transgene interrupted by a selectable marker gene also can be used to obtain transgenic animals with conditional expression of a transgene. For example, a promoter driving expression of the marker/transgene can be either ubiquitous or tissue-specific, which would result in the ubiquitous or tissue-specific expression of the marker in F0 animals (e.g., pigs). Tissue specific activation of the transgene can be accomplished, for example, by crossing a pig that ubiquitously expresses a marker-interrupted transgene to a pig expressing Cre or Flp in a tissue-specific manner, or by crossing a pig that expresses a marker-interrupted transgene in a tissue-specific manner to a pig that ubiquitously expresses Cre or Flp recombinase. Controlled expression of the transgene or controlled excision of the marker allows expression of the transgene.

In some embodiments, the exogenous nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and FLAG™ tag (Kodak, New Haven, Conn.).

Nucleic acid constructs can be methylated using an SssI CpG methylase (New England Biolabs, Ipswich, Mass.). In general, the nucleic acid construct can be incubated with S-adenosylmethionine and SssI CpG-methylase in buffer at 37° C. Hypermethylation can be confirmed by incubating the construct with one unit of HinP1I endonuclease for 1 hour at 37° C. and assaying by agarose gel electrophoresis.

Nucleic acid constructs can be introduced into embryonic, fetal, or adult artiodactyl/livestock cells of any type, including, for example, germ cells such as an oocyte or an egg, a progenitor cell, an adult or embryonic stem cell, a primordial germ cell, a kidney cell such as a PK-15 cell, an islet cell, a beta cell, a liver cell, or a fibroblast such as a dermal fibroblast, using a variety of teclmiques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

In transposon systems, the transcriptional unit of a nucleic acid construct, i.e., the regulatory region operably linked to an exogenous nucleic acid sequence, is flanked by an inverted repeat of a transposon. Several transposon systems, including, for example, Sleeping Beauty (see, U.S. Pat. No. 6,613,752 and U.S. Publication No. 2005/0003542); Frog Prince (Miskey et al. (2003) Nucleic Acids Res. 31:6873); Toll (Kawakami (2007) Genome Biology 8(Suppl. 1):S7; Minos (Pavlopoulos et al. (2007) Genome Biology 8(Suppl. 1):S2); Hsmarl (Miskey et al. (2007)) Mol Cell Biol. 27:4589); and Passport have been developed to introduce nucleic acids into cells, including mice, human, and pig cells. The Sleeping Beauty transposon is particularly useful. A transposase can be delivered as a protein, encoded on the same nucleic acid construct as the exogenous nucleic acid, can be introduced on a separate nucleic acid construct, or provided as an mRNA (e.g., an in vitro-transcribed and capped mRNA).

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in animals have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, and also any necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggyBac).

As used herein, the term nucleic acid refers to both RNA and DNA, including, for example, cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand). The term transgenic is used broadly herein and refers to a genetically modified organism or genetically engineered organism whose genetic material has been altered using genetic engineering techniques. A knockout artiodactyl is thus transgenic regardless of whether or not exogenous genes or nucleic acids are expressed in the animal or its progeny.

### Genetically Modified Animals

Animals may be modified using TALENs or other genetic engineering tools, including recombinase fusion proteins, or various vectors that are known. A genetic modification made by such tools may comprise disruption of a gene. The term disruption of a gene refers to preventing the formation of a functional gene product. A gene product is functional only if it fulfills its normal (wild-type) functions. Disruption of the gene prevents expression of a functional factor encoded by the gene and comprises an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. Materials and methods of genetically modifying animals are further detailed in U.S. Ser. No. 13/404,662 filed Feb. 24, 2012, Ser. No. 13/467,588 filed May 9, 2012, and Ser. No. 12/622,886 filed Nov. 10, 2009 which are hereby incorporated herein by reference for all purposes; in case of conflict, the instant specification is controlling. The term trans-acting refers to processes acting on a target gene from a different molecule (i.e., intermolecular). A trans-acting element is usually a DNA sequence that contains a gene. This gene codes for a protein (or microRNA or other diffusible molecule) that is used in the regulation the target gene. The trans-acting gene may be on the same chromosome as the target gene, but the activity is via the intermediary protein or RNA that it encodes. Inactivation of a gene using a dominant negative generally involves a trans-acting element. The term cis-regulatory or cis-acting means an action without coding for protein or RNA; in the context of gene inactivation, this generally means inactivation of the coding portion of a gene, or a promoter and/or operator that is necessary for expression of the functional gene.

Various techniques known in the art can be used to inactivate genes to make knock-out animals and/or to introduce nucleic acid constructs into animals to produce founder animals and to make animal lines, in which the knockout or nucleic acid construct is integrated into the genome. Such techniques include, without limitation, pronuclear microinjection (U.S. Pat. No. 4,873,191), retrovirus mediated gene transfer into germ lines (Van der Putten et al. (1985) Proc. Natl. Acad. Sci. USA, 82:6148-1652), gene targeting into embryonic stem cells (Thompson et al. (1989) Cell, 56:313-321), electroporation of embryos (Lo (1983) Mol. Cell. Biol., 3:1803-1814), sperm-mediated gene transfer (Lavitrano et al. (2002) Proc. Natl. Acad. Sci. USA, 99:14230-14235; Lavitrano et al. (2006) Reprod. Fert. Develop., 18:19-23), and in vitro transformation of somatic cells, such as cumulus or mammary cells, or adult, fetal, or embryonic stem cells, followed by nuclear transplantation (Wilmut et al. (1997) Nature, 385:810-813; and Wakayama et al. (1998) Nature, 394:369-374). Pronuclear microinjection, sperm mediated gene transfer, and somatic cell nuclear transfer are particularly useful techniques. An animal that is genomically modified is an animal wherein all of its cells have the genetic modification, including its germ line cells. When methods are used that produce an animal that is mosaic in its genetic modification, the animals may be inbred and progeny that are genomically modified may be selected. Cloning, for instance, may be used to make a mosaic animal if its cells are modified at the blastocyst state, or genomic modification can take place when a single-cell is modified. If a particular gene is inactivated by a knock out modification, homozygousity would normally be required. If a particular gene is inactivated by an RNA interference or dominant negative strategy, then heterozygosity is often adequate.

Typically, in pronuclear microinjection, a nucleic acid construct is introduced into a fertilized egg; 1 or 2 cell fertilized eggs are used as the pronuclei containing the genetic material from the sperm head and the egg are visible within the protoplasm. Pronuclear staged fertilized eggs can be obtained in vitro or in vivo (i.e., surgically recovered from the oviduct of donor animals). In vitro fertilized eggs can be produced as follows. For example, swine ovaries can be collected at an abattoir, and maintained at 22-28° C. during transport. Ovaries can be washed and isolated for follicular aspiration, and follicles ranging from 4-8 mm can be aspirated into 50 mL conical centrifuge tubes using 18 gauge needles and under vacuum. Follicular fluid and aspirated oocytes can be rinsed through pre-filters with commercial TL-HEPES (Minitube, Verona, Wis.). Oocytes surrounded by a compact cumulus mass can be selected and placed into TCM-199 OOCYTE MATURATION MEDIUM (Minitube, Verona, Wis.) supplemented with 0.1 mg/mL cysteine, 10 ng/mL epidermal growth factor, 10% porcine follicular fluid, 50 μM 2-mercaptoethanol, 0.5 mg/ml cAMP, 10 IU/mL each of pregnant mare serum gonadotropin (PMSG) and human chorionic gonadotropin (hCG) for approximately 22 hours in humidified air at 38.7° C. and 5% CO₂. Subsequently, the oocytes can be moved to fresh TCM-199 maturation medium, which will not contain cAMP, PMSG or hCG and incubated for an additional 22 hours. Matured oocytes can be stripped of their cumulus cells by vortexing in 0.1% hyaluronidase for 1 minute.

For swine, mature oocytes can be fertilized in 500 μl Minitube PORCPRO IVF MEDIUM SYSTEM (Minitube, Verona, Wis.) in Minitube 5-well fertilization dishes. In preparation for in vitro fertilization (IVF), freshly-collected or frozen boar semen can be washed and resuspended in PORCPRO IVF Medium to 4×10⁵ sperm. Sperm concentrations can be analyzed by computer assisted semen analysis (SPERMVISION, Minitube, Verona, Wis.). Final in vitro insemination can be performed in a 10 μl volume at a final concentration of approximately 40 motile sperm/oocyte, depending on boar. Incubate all fertilizing oocytes at 38.7° C. in 5.0% CO₂ atmosphere for 6 hours. Six hours post-insemination, presumptive zygotes can be washed twice in NCSU-23 and moved to 0.5 mL of the same medium. This system can produce 20-30% blastocysts routinely across most boars with a 10-30% polyspermic insemination rate.

Linearized nucleic acid constructs can be injected into one of the pronuclei. Then the injected eggs can be transferred to a recipient female (e.g., into the oviducts of a recipient female) and allowed to develop in the recipient female to produce the transgenic animals. In particular, in vitro fertilized embryos can be centrifuged at 15,000×g for 5 minutes to sediment lipids allowing visualization of the pronucleus. The embryos can be injected with using an Eppendorf FEMTOJET injector and can be cultured until blastocyst formation. Rates of embryo cleavage and blastocyst formation and quality can be recorded.

Embryos can be surgically transferred into uteri of asynchronous recipients. Typically, 100-200 (e.g., 150-200) embryos can be deposited into the ampulla-isthmus junction of the oviduct using a 5.5-inch TOMCAT® catheter. After surgery, real-time ultrasound examination of pregnancy can be performed.

In somatic cell nuclear transfer, a transgenic artiodactyl cell (e.g., a transgenic pig cell or bovine cell) such as an embryonic blastomere, fetal fibroblast, adult ear fibroblast, or granulosa cell that includes a nucleic acid construct described above, can be introduced into an enucleated oocyte to establish a combined cell. Oocytes can be enucleated by partial zona dissection near the polar body and then pressing out cytoplasm at the dissection area. Typically, an injection pipette with a sharp beveled tip is used to inject the transgenic cell into an enucleated oocyte arrested at meiosis 2. In some conventions, oocytes arrested at meiosis-2 are termed eggs. After producing a porcine or bovine embryo (e.g., by fusing and activating the oocyte), the embryo is transferred to the oviducts of a recipient female, about 20 to 24 hours after activation. See, for example, Cibelli et al. (1998) Science, 280:1256-1258 and U.S. Pat. No. 6,548,741. For pigs, recipient females can be checked for pregnancy approximately 20-21 days after transfer of the embryos.

Spermatogonial stem cells offer a second method for genetic modification of livestock. Genetic modification or gene edits can be executed in vitro in spermatogonal stem cells isolated from donor testes. Modified cells are transplanted into germ-cell depleted testes of a recipient. Implanted spermatogonial stem cells produce sperm that carry the genetic modification(s) that can be used for breeding via artificial insemination (AI) or in vitro fertilization (IVF) to derive founder animals.

Standard breeding techniques can be used to create animals that are homozygous for the exogenous nucleic acid from the initial heterozygous founder animals. Homozygosity may not be required, however. Transgenic pigs described herein can be bred with other pigs of interest.

In some embodiments, a nucleic acid of interest and a selectable marker can be provided on separate transposons and provided to either embryos or cells in unequal amount, where the amount of transposon containing the selectable marker far exceeds (5-10 fold excess) the transposon containing the nucleic acid of interest. Transgenic cells or animals expressing the nucleic acid of interest can be isolated based on presence and expression of the selectable marker. Because the transposons will integrate into the genome in a precise and unlinked way (independent transposition events), the nucleic acid of interest and the selectable marker are not genetically linked and can easily be separated by genetic segregation through standard breeding. Thus, transgenic animals can be produced that are not constrained to retain selectable markers in subsequent generations, an issue of some concern from a public safety perspective.

Once transgenic animal have been generated, expression of an exogenous nucleic acid can be assessed using standard techniques. Initial screening can be accomplished by Southern blot analysis to determine whether or not integration of the construct has taken place. For a description of Southern analysis, see sections 9.37-9.52 of Sambrook et al., 1989, Molecular Cloning, A Laboratory Manual, second edition, Cold Spring Harbor Press, Plainview; N.Y. Polymerase chain reaction (PCR) techniques also can be used in the initial screening. PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. PCR is described in, for example PCR Primer: A Laboratory Manual, ed. Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplified. See, for example, Lewis (1992) Genetic Engineering News, 12:1; Guatelli et al. (1990) Proc. Natl. Acad Sci. USA, 87:1874; and Weiss (1991) Science, 254:1292. At the blastocyst stage, embryos can be individually processed for analysis by PCR, Southern hybridization and splinkerette PCR (see, e.g., Dupuy et al. Proc Natl Acad Sci USA (2002) 99:4495).

Expression of a nucleic acid sequence encoding a polypeptide in the tissues of transgenic pigs can be assessed using techniques that include, for example, Northern blot analysis of tissue samples obtained from the animal, in situ hybridization analysis, Western analysis, immunoassays such as enzyme-linked immunosorbent assays, and reverse-transcriptase PCR (RT-PCR).

### Interfering RNAs

A variety of interfering RNA (RNAi) are known. Double-stranded RNA (dsRNA) induces sequence-specific degradation of homologous gene transcripts. RNA-induced silencing complex (RISC) metabolizes dsRNA to small 21-23-nucleotide small interfering RNAs (siRNAs). RISC contains a double stranded RNAse (dsRNase, e.g., Dicer) and ssRNase (e.g., Argonaut 2 or Ago2). RISC utilizes antisense strand as a guide to find a cleavable target. Both siRNAs and microRNAs (miRNAs) are known. A method of disrupting a gene in a genetically modified animal comprises inducing RNA interference against a target gene and/or nucleic acid such that expression of the target gene and/or nucleic acid is reduced.

For example the exogenous nucleic acid sequence can induce RNA interference against a nucleic acid encoding a polypeptide. For example, double-stranded small interfering RNA (siRNA) or small hairpin RNA (shRNA) homologous to a target DNA can be used to reduce expression of that DNA. Constructs for siRNA can be produced as described, for example, in Fire et al. (1998) Nature, 391:806; Romano and Masino (1992) Mol. Microbiol., 6:3343; Cogoni et al. (1996) EMBO 1, 15:3153; Cogoni and Masino (1999) Nature, 399:166; Misquitta and Paterson (1999) Proc. Natl. Acad. Sci. USA, 96:1451; and Kennerdell and Carthew (1998) Cell, 95:1017. Constructs for shRNA can be produced as described by McIntyre and Fanning (2006) BMC Biotechnology, 6:1. In general, shRNAs are transcribed as a single-stranded RNA molecule containing complementary regions, which can anneal and form short hairpins.

The probability of finding a single, individual functional siRNA or miRNA directed to a specific gene is high. The predictability of a specific sequence of siRNA, for instance, is about 50% but a number of interfering RNAs may be made with good confidence that at least one of them will be effective:

Embodiments include an in vitro cell, an in vivo cell, and a genetically modified animal such as a livestock animal that express an RNAi directed against a gene, e.g., a gene selective for a developmental stage. The RNAi may be, for instance, selected from the group consisting of siRNA, shRNA, dsRNA, RISC and miRNA.

### Inducible Systems

An inducible system may be used to control expression of a gene. Various inducible systems are known that allow spatiotemporal control of expression of a gene. Several have been proven to be functional in vivo in transgenic animals. The term inducible system includes traditional promoters and inducible gene expression elements.

An example of an inducible system is the tetracycline (tet)-on promoter system, which can be used to regulate transcription of the nucleic acid. In this system, a mutated Tet repressor (TetR) is fused to the activation domain of herpes simplex virus VP16 trans-activator protein to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent.

The tetracycline-inducible system and the Cre/loxP recombinase system (either constitutive or inducible) are among the more commonly used inducible systems. The tetracycline-inducible system involves a tetracycline-controlled transactivator (tTA)/reverse tTA (rtTA). A method to use these systems in vivo involves generating two lines of genetically modified animals. One animal line expresses the activator (tTA, rtTA, or Cre recombinase) under the control of a selected promoter. Another set of transgenic animals express the acceptor, in which the expression of the gene of interest (or the gene to be modified) is under the control of the target sequence for the tTA/rtTA transactivators (or is flanked by loxP sequences). Mating the two strains of mice provides control of gene expression.

The tetracycline-dependent regulatory systems (tet systems) rely on two components, i.e., a tetracycline-controlled transactivator (tTA or rtTA) and a tTA/rtTA-dependent promoter that controls expression of a downstream cDNA, in a tetracycline-dependent manner. In the absence of tetracycline or its derivatives (such as doxycycline), tTA binds to tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. Administration of tetracycline or its derivatives allows temporal control of transgene expression in vivo. rtTA is a variant of tTA that is not functional in the absence of doxycycline but requires the presence of the ligand for transactivation. This tet system is therefore termed tet-ON. The tet systems have been used in vivo for the inducible expression of several transgenes, encoding, e.g., reporter genes, oncogenes, or proteins involved in a signaling cascade.

The Cre/lox system uses the Cre recombinase, which catalyzes site-specific recombination by crossover between two distant Cre recognition sequences, i.e., loxP sites. A DNA sequence introduced between the two loxP sequences (termed foxed DNA) is excised by Cre-mediated recombination. Control of Cre expression in a transgenic animal, using either spatial control (with a tissue- or cell-specific promoter) or temporal control (with an inducible system), results in control of DNA excision between the two loxP sites. One application is for conditional gene inactivation (conditional knockout). Another approach is for protein over-expression, wherein a foxed stop codon is inserted between the promoter sequence and the DNA of interest. Genetically modified animals do not express the transgene until Cre is expressed, leading to excision of the floxed stop codon. This system has been applied to tissue-specific oncogenesis and controlled antigene receptor expression in B lymphocytes. Inducible Cre recombinases have also been developed. The inducible Cre recombinase is activated only by administration of an exogenous ligand. The inducible Cre recombinases are fusion proteins containing the original Cre recombinase and a specific ligand-binding domain. The functional activity of the Cre recombinase is dependent on an external ligand that is able to bind to this specific domain in the fusion protein.

Embodiments include an in vitro cell, an in vivo cell, and a genetically modified animal such as a livestock animal that comprise a gene under control of an inducible system. The genetic modification of an animal may be genomic or mosaic. The inducible system may be, for instance, selected from the group consisting of Tet-On, Tet-Off, Cre-lox, and Hif1alpha. An embodiment is a gene set forth herein.

### Founder Animals, Animal Lines, Traits, and Reproduction

Founder animals may be produced by cloning and other methods described herein. The founders can be homozygous for a genetic modification, as in the case where a zygote or a primary cell undergoes a homozygous modification. Similarly, founders can also be made that are heterozygous. The founders may be genomically modified, meaning that all of the cells in their genome have undergone modification. Founders can be mosaic for a modification, as may happen when vectors are introduced into one of a plurality of cells in an embryo, typically at a blastocyst stage. Progeny of mosaic animals may be tested to identify progeny that are genomically modified. An animal line is established when a pool of animals has been created that can be reproduced sexually or by assisted reproductive techniques, with heterogeneous or homozygous progeny consistently expressing the modification.

In livestock, many alleles are known to be linked to various traits such as production traits, type traits, workability traits, and other functional traits. Artisans are accustomed to monitoring and quantifying these traits, e.g., Visscher et al., Livestock Production Science, (1994) 40:123-137, U.S. Pat. No. 7,709,206, US 2001/0016315, US 2011/0023140, and US 2005/0153317. An animal line may include a trait chosen from a trait in the group consisting of a production trait, a type trait, a workability trait, a fertility trait, a mothering trait, and a disease resistance trait. Further traits include expression of a recombinant gene product. Embodiments include selecting an animal with one or more such traits, or a genetically introduced trait, and modifying its genome to include marked sperm.

### Recombinases

Embodiments of the invention include administration of a targeted nuclease system with a recombinase (e.g., a RecA protein, a Rad51) or other DNA-binding protein associated with DNA recombination. A recombinase forms a filament with a nucleic acid fragment and, in effect, searches cellular DNA to find a DNA sequence substantially homologous to the sequence. For instance a recombinase may be combined with a nucleic acid sequence that serves as a template for HDR. The recombinase is then combined with the HDR template to form a filament and placed into the cell. The recombinase and/or HDR template that combines with the recombinase may be placed in the cell or embryo as a protein, an mRNA, or with a vector that encodes the recombinase. The disclosure of US Pub 2011/0059160 (U.S. Ser. No. 12/869,232) is hereby incorporated herein by reference for all purposes; in case of conflict, the specification is controlling. The term recombinase refers to a genetic recombination enzyme that enzymatically catalyzes, in a cell, the joining of relatively short pieces of DNA between two relatively longer DNA strands. Recombinases include Cre recombinase, Hin recombinase, RecA, RAD51, Cre, and FLP. Cre recombinase is a Type I topoisomerase from P1 bacteriophage that catalyzes site-specific recombination of DNA between loxP sites. Hin recombinase is a 21 kD protein composed of 198 amino acids that is found in the bacteria Salmonella. Hin belongs to the serine recombinase family of DNA invertases in which it relies on the active site serine to initiate DNA cleavage and recombination. RAD51 is a human gene. The protein encoded by this gene is a member of the RAD51 protein family which assists in repair of DNA double strand breaks. RAD51 family members are homologous to the bacterial RecA and yeast Rad51. Cre recombinase is an enzyme that is used in experiments to delete specific sequences that are flanked by loxP sites. FLP refers to Flippase recombination enzyme (FLP or Flp) derived from the 2μ plasmid of the baker's yeast Saccharomyces cerevisiae.

Herein, “RecA” or “RecA protein” refers to a family of RecA-like recombination proteins having essentially all or most of the same functions, particularly: (i) the ability to position properly oligonucleotides or polynucleotides on their homologous targets for subsequent extension by DNA polymerases; (ii) the ability topologically to prepare duplex nucleic acid for DNA synthesis; and, (iii) the ability of RecA/oligonucleotide or RecA/polynucleotide complexes efficiently to find and bind to complementary sequences. The best characterized RecA protein is from E. coli; in addition to the original allelic form of the protein a number of mutant RecA-like proteins have been identified, for example, RecA803. Further, many organisms have RecA-like strand-transfer proteins including, for example, yeast, Drosophila, mammals including humans, and plants. These proteins include, for example, Rec1, Rec2, Rad51, Rad51B, Rad51C, Rad51D, Rad51E, XRCC2 and DMC1. An embodiment of the recombination protein is the RecA protein of E. coli. Alternatively, the RecA protein can be the mutant RecA-803 protein of E. coli, a RecA protein from another bacterial source or a homologous recombination protein from another organism.

### Compositions and Kits

The present invention also provides compositions and kits containing, for example, nucleic acid molecules encoding site-specific endonucleases, CRISPR, Cas9, ZNFs, TALENs, polypeptides of the same, compositions containing such nucleic acid molecules or polypeptides, or engineered cell lines. An HDR may also be provided that is effective for introgression of an indicated allele. Such items can be used, for example, as research tools, or therapeutically.

### EXAMPLES

Materials and methods, including making of TALENs, are generally as described in U.S. Ser. No. 13/594,694 filed Aug. 24, 2012, unless otherwise indicated.

### Example 1

### TALENs for Y-Chromosome Modification

Transfection—Fibroblasts are cultured and transfected by nucleofection as previously described. (Carlson et al., 2011) Transposon components total 1 μg in the Experiments. For Homology-Dependent Repair (HDR) analysis, the best performing condition for Double-Strand-Break (DSB) induction are chosen and repair template is added at equal, 3 and 10 fold excess to TALEN plasmid. Cell culture—Isolation of individual colonies is conducted by selection in 96-well plates at pre-determined densities to result in colonies in 30-50% of wells. Indel detection populations—Primers flanking the target sites are designed to result in amplicons ˜500 bp. PCR amplicons are treated with SURVEYOR® Nuclease (Transgenomic, Omaha Nebr.) as suggested, and resolved on 8-10% polyacrylamide gels. A portion of amplicons from indel positive blastocysts are cloned and sequenced to characterize the mutation. Indel detection colonies—Primers flanking the target site as used above are used for amplification using the High Resolution Melt analysis ciPCR master mix (Invitrogen) and melting curves analysis will be conducted. The variation in melt profile of the real time PCR product will distinguish clones carrying TALEN induced mutation from wild type sequence. Normal variation in the melting temperature of amplicons derived non-transfected control cells will be used as a reference. Amplicons with melt profiles outside of the normal variation are cloned and sequenced to characterize mutations. Y-Targeting detection—PCR assays are developed with a primer outside of the homology arms and one within to result in a product only possible if homologous recombination has occurred. PCR-positive colonies are validated by Whole Genome Amplification Southern blotting. WGA Southern Blotting to confirm Y-targeting—WGA is performed on individual clones using half reactions of the REPLI-g Mini Kit (Qiagen, Valencia, Calif.) according to the “Amplification of Blood or Cells” protocol. Probes for Southern Blotting are hybridized to validate 5′ and 3′ junctions of targeted cells. FACS—Fresh semen is prepared for sorting of X- and Y-bearing sperm cells by placing 15 million spermatozoa in 1 ml of BTS with Hoechst 33342 added to a concentration of 6.25 uM. This preparation is incubated for 45 min at 35° C. X- and Y-bearing sperm are sorted by DNA content using a modified flow cytometer with standard modifications for sperm sorting. (Johnson et al., 1987; Johnson and Pinkel, 1986) Accuracy of sorted populations is determined by quantitative PCR for X and Y targets. Serum hormone measurements—Blood serum levels of testosterone and FSH are evaluated using commercially ELISA kits from Endocrine Technologies. (Newark, Calif.).

Four TALEN pairs were made that are directed against two candidate loci for Y chromosome gene addition (FIG. 4). The first candidate is located 1.7 kb 3′ of SRY, beyond the two highest ranking poly-adenylation signals. A second candidate locus is the Y-specific intron of the AMELY gene. These loci are predicted to lie outside of the pseudoautosomal boundary of SSCY based on comparison with cattle and pig:cattle comparative gene mapping data. (Quilter et al., 2002; Van Laere et al., 2008) As such, they are not capable of undergoing recombination with SSCX or autosomes and thus expected to be maintained on SSCY across numerous generations. Three of four TALENs pairs tested revealed high activity (FIG. 4).

### Example 2

### Isolation of Mono- and Bi-Allelic KO Clones

Carlson et al. 2012 described modification of target genes in livestock wherein transgenic primary fibroblasts were effectively expanded and isolated as colonies when plated with non-transgenic fibroblasts (feeder-cells) at standard densities (>150 cells/cm²) and subjected to drug selection using the transposon co-selection technique applied above (Carlson et al. (2011) Transgenic Res., 20:1125 and US Pub 2012/0220037 filed May 9, 2012). These techniques are useful for making genetic changes to somatic cells that can be used to clone animals.

As an example, puromycin resistant colonies for cells treated with six TALEN pairs were isolated and their genotypes evaluated. In general, the proportion of indel positive clones was similar to predictions made based on day 3 modification levels. Bi-alleic knockout clones were identified for 6 of 7 different TALEN pairs, occurring in up to 35 percent of indel positive cells. In the majority of examples, indels were homozygous (same indel on each allele) rather than unique indels on each allele suggesting that sister chromatid-templated repair is common. Notably, among modified clones, the frequency of bi-alleic modification (17-60%) for the majority of TALEN pairs exceed predictions based on day 3 modification levels (10-17%) if chromosome cleavages are treated as independent events.

### Example 3

### TALEN Mediated DNA Cleavage as a Target for HDR in Livestock Cells

TALEN pair (LDLR4.2) targeted to the fourth exon of the swine low density lipoprotein receptor (LDLR) gene was co-transfected with the supercoiled plasmid Ldlr-E4N-stop, which contains homology arms corresponding to the swine LDLR gene and a gene-trap enabling expression of Neomycin phosphotransferase upon HDR. After 3 days of culture PCR analysis revealed that, even without antibiotic selection, a band corresponding to an HDR event could be detected at the targeted locus at 30° C. Selection of populations of cultured cells for 14 days with geneticin (G418) resulted in significant enrichment of HDR cells.

### Example 4

### Single Stranded DNA for Templating

Tan et al. 2013 described use of single stranded DNA of template-driven modification of genes. Single stranded oligodeoxynucleotides (ssODNs) are an effective template for TALEN stimulated HR. Loci were targeted to introgress the 11 base pair Belgian Blue cattle mutation into Wagyu cells. Two 76 base pair ssODNs were designed to mimic either the sense or antisense strand of the BB GDF8 gene including the 11 base pair deletion. Four micrograms of TALEN encoding plasmids were transfected into Wagyu cells, and 0.3 nMol of ssODNs were either co-transfected with TALENS (N) or delivered 24 hours after TALEN nucleofection by either MirusLT1 (M) reagent or Lipofectamine LTX reagent (L). Semi-quantitative PCR at day three indicated an allele conversion frequency of up to 5% in conditions where ssODNs were delivered with LIPOFECTAMINE LTX reagent 24 hours after TALEN transfection. No difference in PCR signal was observed between sense and antisense ssODNs designed against the target.

### Example 5

### Alleles Introduced into Pig (Ossabaw) Cells Using Oligo HDR

Tan et al. (2013) describe modifying cells with a combination of mRNA encoded TALENs and single-stranded oligonucleotides to place an allele that occurs naturally in one species to another species (interspecific migration). Piedmontese GFD8 SNP C313Y, were chosen as an example and was introduced into Ossabow swine cells. No markers were used in these cells at any stage. A similar peak in HDR was observed between pig and cattle cells at 0.4 nmol ssODN, (not shown) however, HDR was not extinguished by higher concentrations of ssODN in Ossabaw fibroblasts.

### Example 6

### CRISPR/Cas9 Design and Production

Gene specific gRNA sequences were cloned into the Church lab gRNA vector (Addgene ID: 41824) according their methods. The Cas9 nuclease was provided either by co-transfection of the hCas9 plasmid (Addgene ID: 41815) or mRNA synthesized from RCIScript-hCas9. This RCIScript-hCas9 was constructed by sub-cloning the XbaI-AgeI fragment from the hCas9 plasmid (encompassing the hCas9 cDNA) into the RCIScript plasmid. Synthesis of mRNA was conducted as above except that linearization was performed using KpnI.

### Example 7

### CRISPR/Cas9

CRISPR/Cas9 mediated HDR was used to introgress the p65 S531P mutation from warthogs into conventional swine. Referring to FIG. 5, at Panel a) The S531P missense mutation is caused by a T-C transition at nucleotide 1591 of porcine p65 (RELA). The S-P HDR template includes the causative TC transition mutation (oversized text) which introduces a novel XmaI site and enables RFLP screening. Two gRNA sequences (P65_G1S and P65_G2A) are shown along with the p65.8 TALENs used in previous experiments. At panel b) Landrace fibroblasts were transfected with S-P-HDR oligos (2 μM), two quantities of plasmid encoding hCas9 (0.5 μg vs. 2.0 μg); and five quantities of the G2A transcription plasmid (0.05 to 1.0 μg). Cells from each transfection were split 60:40 for culture at 30 and 37° C. respectively for 3 days before prolonged culture at 37° C. until day 10. Surveyor assay revealed activity ranging from 16-30%. Panels c and d) RFLP analysis of cells sampled at days 3 and 10. Expected cleavage products of 191 and 118 bp are indicated by black arrows. Despite close proximity of the DSB to the target SNP, CRISPR/Cas9 mediated HDR was less efficient than TALENs for introgression of S531P. Individual colonies were also analyzed using each gRNA sequence.

### Example 8

### CRISPR/Cas9

Comparison of TALENs and CRISPR/Cas9 mediated HDR at porcine APC. Referring to FIG. 6, at panel a) APC 14.2 TALENs and the gRNA sequence APC 14.2 G1a are shown relative to the wild type APC sequence. Below, the HDR oligo is shown which delivers a 4 bp insertion (see text) resulting in a novel HindIII site. Pig fibroblasts transfected with 2 μM of oligo HDR template, and either 1 μg TALEN mRNA, 1 μg each plasmid DNA encoding hCas9 and the gRNA expression plasmid; or 1 μg mRNA encoding hCas9 and 0.5 μg of gRNA expression plasmid, were then split and cultured at either 30 or 37° C. for 3 days before expansion at 37° C. until day 10. Panel b) Charts displaying RFLP and Surveyor assay results. As previously determined TALEN stimulated HDR was most efficient at 30° C., while CRISPR/Cas9 mediated HDR was most effective at 37° C. For this locus, TALENs were more effective than the CRISPR/Cas9 system for stimulation of HDR despite similar DNA cutting frequency measured by SURVEYOR assay. In contrast to TALENs, there was little difference in HDR when hCas9 was delivered as mRNA versus plasmid.

### Example 9

### Targeting the Y-Chromosome

A combination of TALENs and plasmid homology cassettes were used to target the mCaggs-EGFP cassette to the Y-chromosome. For the purposes of this experiment, the positive orientation is when both the transgene cassette and the endogenous gene (SRY or AMELY) are in the same orientation, the negative orientation is when they are in opposite orientation. One microgram of TALEN mRNA plus 500 ng of the homology cassette was mixed with 600,000 cells in a single 100 ul electroporation. Cells were transfected using the NEON electroporation system (Life Technologies), cultured for 3 days at 30° C., and plated at low density for derivation of single cell derived colonies. Colonies were analyzed for correct targeting of the Y chromosome by junction PCR using one primer outside of the homology arms and a second primer within the transgene cassette. Several colonies were positive for the expected amplicon. FIG. 8 is a summary of the results shown in FIG. 7. Clones positive for Y-targeting ranged from 6-24 percent. The orientation of the transgene cassette appeared to have some effect on the efficiency of Y-targeting.

### Example 10

### Short Homology Targeting of the Y Chromosome

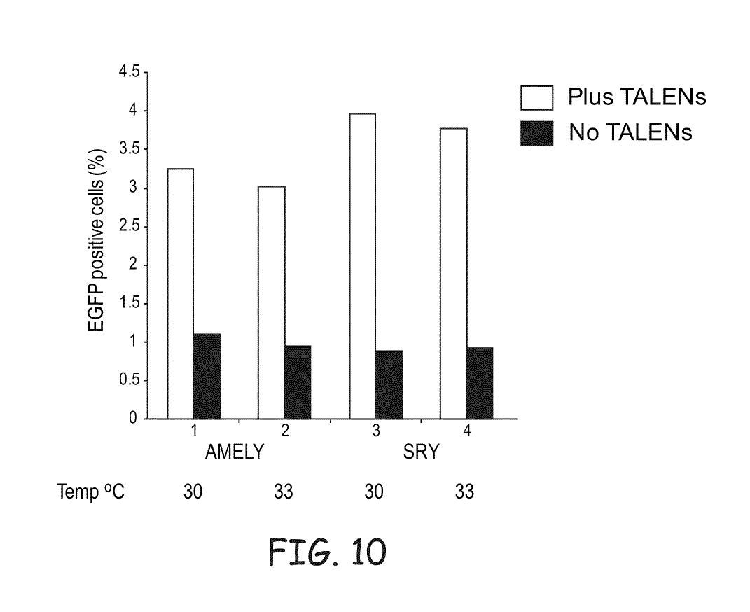

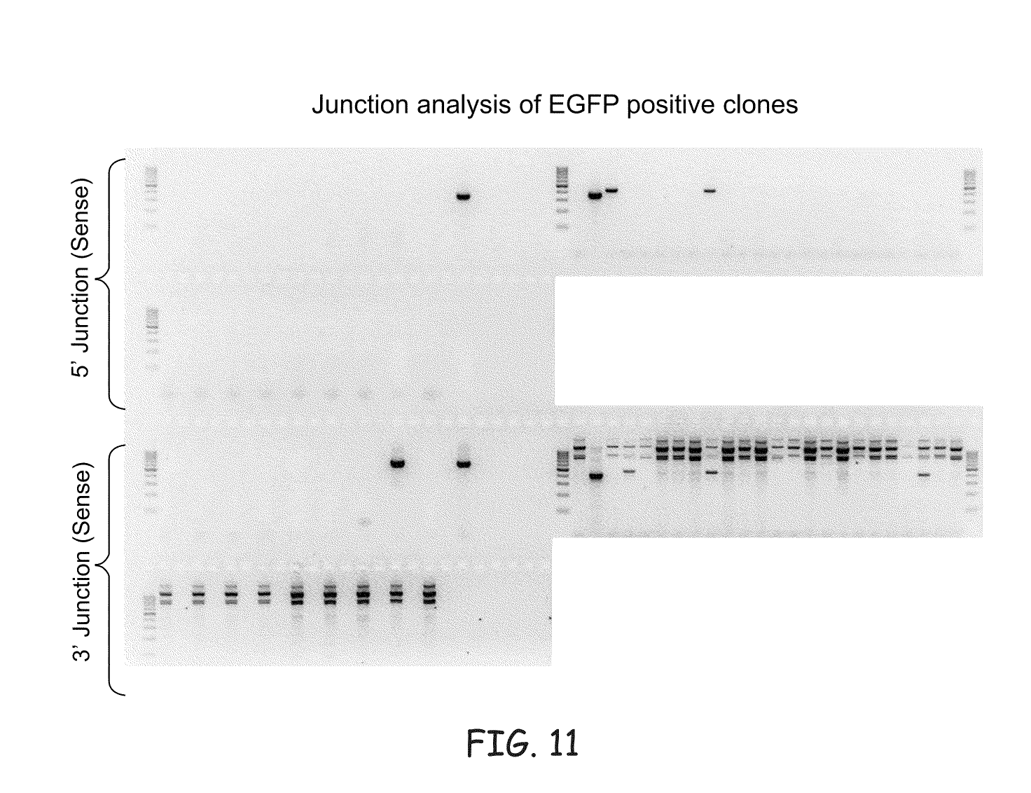

As an alternative to plasmid homology cassettes, linear templates with short (50-100 bp) homology arms were developed to target AMELY and SRY sites. The homology templates were created by PCR amplification of the ubiquitin EGFP cassette using primers that bound to the 5′ and 3′ end of the cassette and included a tail corresponding to the sequence 5′ and 3′ of the presumptive TALEN induced double strand break as described in Orlando et al. 2010 (NAR; 2010 August; 38(15)). The primers included phosporthioate linkages between the first two nucleotides to inhibit degradation by endogenous nucleases. Two micrograms of TALEN mRNA (or none as control) and 1 ug of short homology template specific to each site was included in a typical 100 ul electroporation. After electroporation, the cells were divided for culture at either 30 or 33° C. for three days, followed by junction PCR to test for Y-targeting. Cells cultured at 30 or 33° C. were positive for Y-targeting at both the 5′ and 3′ junction, though product intensity suggests Y-targeting is more efficient at 30° C. For each site, amplicons corresponding to correct Y-targeting was dependent on TALENs, note the top band of the SRY 3′ junction is non-specific background signal. Cell populations cultured for 14 days post-transfection should no longer express non-integrated templates. FACs for EGFP was conducted on day 14 populations to determine if the combination of TALENs plus the short homology template, versus template alone, increases the proportion of EGFP positive cells. Indeed, EGFP positive cells were ˜3-fold enriched when TALENs were included and little temperature effect was observed (FIG. 10). Individual EGFP positive colonies were genotyped for Y-targeting. For AMELY, 0/5 (0%) and 2/5 (20%) of EGFP positive colonies were also positive for Y-targeting from cells initially cultured at 30 or 33° C. respectively (FIG. 11). For SRY, 5/24 (21%) and 0/9 (0%) of EGFP positive colonies were also positive for Y-targeting from cells initially cultured at 30 or 33° C. respectively (FIG. 11).

### Example 11

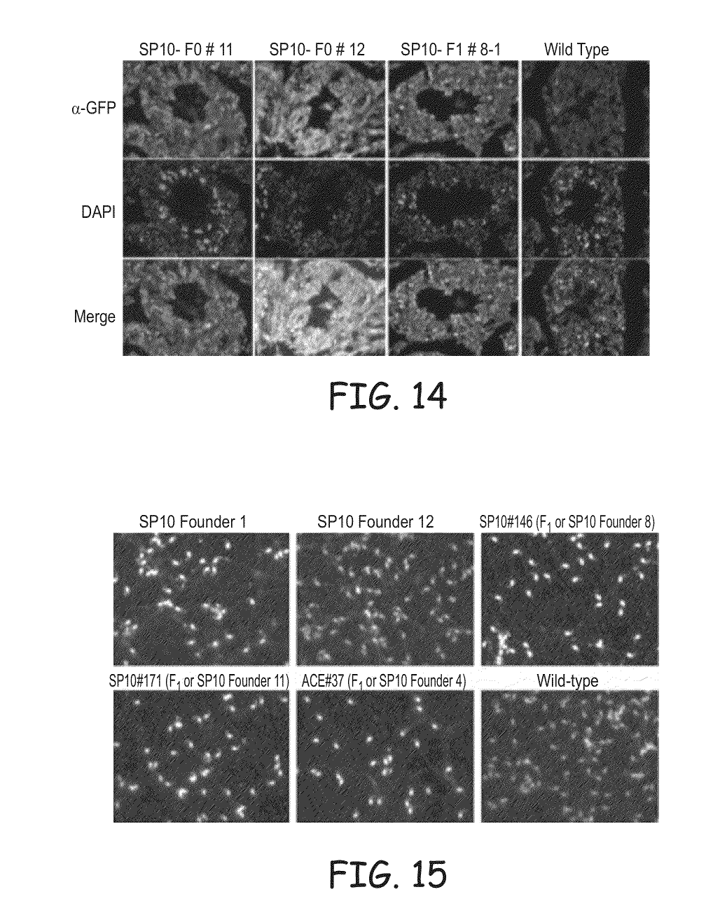

A series of three Sleeping Beauty transposons were created to carry a presumptive cis-restricted transgene under the direction of either the porcine ACE, CK-15 or SP10 promoters, all originally cloned by Applicant's team based on comparative data with mice. Mice were produced by pronuclear injection of Sleeping Beauty transposons as described in Carlson et al., 2011 (FIG. 12). Transgenic mice were subsequently analyzed in the F0 or F1 first by qRT-PCR for the EGFP transgene in the testes (FIG. 13). While no significant expression was observed in either ACE or CK-15 transgenics, there was significant expression in both F0 and F1 mice with the SP 10 promoter. This result was not expected as the orthologous murine ACE and CK-15 promoters reliably express in mouse spermatigonia (Langford, K G et. al. 1991; Albanesi et al., 1996). The localization of EGFP expression in the testes was analyzed by immunohistochemical (IHC) detection. Signal was concentrated in regions of the seminiferous tubule matching the normal progression of spermatogenesis (FIG. 14). Finally, epididymal sperm was analyzed for expression of EGFP by IHC. Consistent with the results of the qPCR, signal was detected in only sperm from SP10 founders. It was observed that founder SP10-11 had multiple copies of the cisX transgene, indicated by high F1 transmission frequency

### Immunohistochemistry

### Testis Sample Preparation.

Testis tissues harvested from euthanized male mice were bisected once on the longitudinal side to form two halves approximately 40 μm long×40 μm in diameter. Tissues were placed in 4% PFA/10% sucrose in PBS, pH 7.2 overnight and embedded in OCT compound at −70° C. Embedded tissues were store thereafter at −80° C. Cryosections were cut at 6 μm, air dried on charged slides for at least 30 minutes, and post-fixed in cold acetone for 5 minutes.

### Staining.

Slides were processed for antigen retrieval in citrate buffer, pH 6.1 (Dako) using the pressure cooker method. Slides were permeabilized in 0.125% Triton-X 100 in PBS for 5 minutes and washed once for 5 minutes in PBS before being immersed in a blocking buffer [2.5% goat serum (DGS), 2.5% fetal bovine serum (FBS) in PBS] for 50 minutes. Slides were washed once in PBS for 5 minutes and 200 μl of primary antibody, polyclonal rabbit anti-GFP (Abeam ab290), diluted 1:200 in PBS with 1.25% each DOS and FBS added. Slides serving as negative (secondary) controls received 200 μl of blocking buffer. Slides were incubated overnight in a humidified chamber at 4° C. Slides were washed in PBS (5 changes, 5 minutes each) and 200 μl of the secondary antibody, goat anti-rabbit IgG F(ab′)₂ conjugated to ALEXAFLUOR 594 (Invitrogen) at 4 μg/ml was added. Slides were incubated at room temperature for 1 hour in the dark. Slides were washed in PBS (five changes, 5 minutes each) and mounted in aqueous mounting media containing DAPI and examined as described in microscopy.

### Spermatozoa Sample Preparation.

Aliquots of spermatozoa were harvested from the epididymis of euthanized male mice into standard seminal cryopreservation media and stored at −80° C. Twenty microliters of each sample was washed in 1 ml of PBS (800×g., 10 minutes). Spermatozoa were resuspended in 200 μl of PBS. 12 mm poly-D-lysine coated coverslips (BD BIOCOAT/Corning) were placed in wells of a 24 well plate. 50 μl of resuspended spermatozoa was spread on each coverslip. Coverslips were dried down at 37° C. and fixed in 100% methanol for 35 seconds. Coverslips were allowed to dry and stored at −20° C.

### Staining.

Spermatozoa samples fixed to coverslips were permeabilized additionally for 40 minutes in 0.1% TRITON-X 100 in PBS and blocked for 1 hour in PBS containing 2.5% each DGS and FBS. Coverslips were washed once in 1 ml PBS for 5 minutes and 200 μl of primary antibody, polyclonal rabbit anti-GFP (Abeam, ab290), diluted 1:200 in PBS with 1.25% each DGS and FBS added to each well. Coverslips serving as negative (secondary) controls received 200 μl of blocking buffer. Coverslips contained in the 24 well plate were incubated overnight at 4° C. Coverslips were washed in PBS (5 changes, 5 minutes each) and 200 μl of the secondary antibody, goat anti-rabbit IgG F(ab′)₂ conjugated to ALEXAFLUOR 594 (Invitrogen) at 4 μg/ml was added to each well. Slides were incubated at room temperature for 1 hour in the dark. Slides were washed in PBS (five changes, 5 minutes each). Coverslips were carefully extracted from the wells and mounted onto slides using 10 μl of aqueous mounting media containing DAPI and examined as described in microscopy.

### Microscopy.

Slides containing testis and spermatozoa samples were examined on a Nikon E800 upright microscope equipped with a motorized-stage configured to image in transmitted light, DIC, epi-fluorescence, epi-polarization, and hyperspectral modes using a Photometrics COOLSNAP MYO monochrome camera and Nikon ELEMENTS AR software installed on a HP computer (64 bit). Fluorescence signal, illuminated by an X-Cite 120 LED lamp source, was obtained via a U/B/G (triple DAPI/FITC/Texas Red) narrowband cubes. Tissue section images (FIG. 13) were collected for each sample using a 20× objective at identical exposure times (16 second for Texas Red/30 seconds DAPI) using the Nikon Elements AR software while spermatozoa images (FIG. 14) were collected using a 40× objective and exposure times of 20 seconds in each channel.

### Quantitative PCR

### Testis Sample Preparation.

Testis tissues harvested from euthanized male mice were bisected once on the longitudinal side to form two halves approximately 40 μm long×40 μm in diameter. One half of one testis was placed in RNALATER (Invitrogen) and stored at −80° C.

RNA Isolation and cDNA Generation.

Testis sample was removed from RNAlater and disrupted using a polytron apparatus in RLT buffer containing β-mercaptoethanol following manufacturer's directions (Qiagen, RNEASY). Total RNA was purified using the RNeasy kit. One microgram of total RNA was converted into cDNA using a two step cDNA kit (Quanta Biosciences) that incorporates a proprietary mix of oligo dT and random hexomers as primers.

Quantitative PCR (qPCR).

Equal amounts of cDNA were used as template in qPCR using SYBR green as a reporter. Reactions were run on a BioRad CFX Connect Real-time System using primers to EGFP and integrin alpha 6 (Itga6), a marker of spermatogonial stem cells, for normalization.

SEQUENCES
Cis-X cassette-EGFP is underlined, the sequence
preceding and post EGFP is Smok1 5 and 3′ UTR.
(SEQ ID NO: 11)
TGTGTGTTTGGGAGGAGCTTGTGTGTGTGAGTTGTGTTTTAAGTTT
ATTTGCGTGTGAGTACCTTTGGGTTTTTGTGTGTGTCTGTGTGTGT
TTGTGTGTGTATAACTGTGGGTGACTGTAAGTGCACCTGTGTGTTT
GTACGTGAGTGTGTAAGACTGTGTGTGTGCACAAGAGCGTGTGTAG
GTGCACGTGTTGTAGGTGTGAGAACACCTGTTGTGTTTAGGCCATC
AGTCAGCTTGGTCATTGTTTCTAAGGTAGCATTTATACTTTGTTAC
CTCAAGTGGGCTCTGGGAGTCAACAGAAGTCAGAAAAGCTCAGATC
CAAGCCCCCTTTTTCTGACCTCGAGACCATGGCCAGGAACGTCACG
AAACGGAAAAACAGGTGCCGCGGACATCAGAAGGCTATTTACAAGA
AAAAGTCTGTGAGCAAGGGCGAGGAGCTGTTCACCGGGGTGGTGCC
CATCCTGGTCGAGCTGGACGGCGACGTAAACGGCCACAAGTTCAGC
GTGTCCGGCGAGGGCGAGGGCGATGCCACCTACGGCAAGCTGACCC
TGAAGTTCATCTGCACCACCGGCAAGCTGCCCGTGCCCTGGCCCAC
CCTCGTGACCACCCTGACCTACGGCGTGCAGTGCTTCAGCCGCTAC
CCCGACCACATGAAGCAGCACGACTTCTTCAAGTCCGCCATGCCCG
AAGGCTACGTCCAGGAGCGCACCATCTTCTTCAAGGACGACGGCAA
CTACAAGACCCGCGCCGAGGTGAAGTTCGAGGGCGACACCCTGGTG
AACCGCATCGAGCTGAAGGGCATCGACTTCAAGGAGGACGGCAACA
TCCTGGGGCACAAGCTGGAGTACAACTACAACAGCCACAACGTCTA
TATCATGGCCGACAAGCAGAAGAACGGCATCAAGGTGAACTTCAAG
ATCCGCCACAACATCGAGGACGGCAGCGTGCAGCTCGCCGACCACT
ACCAGCAGAACACCCCCATCGGCGACGGCCCCGTGCTGCTGCCCGA
CAACCACTACCTGAGCACCCAGTCCGCCCTGAGCAAAGACCCCAAC
GAGAAGCGCGATCACATGGTCCTGCTGGAGTTCGTGACCGCCGCCG
GGATCACTCTCGGCATGGACGAGCTGTACAAGTAAAGTCGAGATAT
GTCGACGACACAGGAAGGGTGTCAGGAGAACGAGCATTCGGCTCGG
CACAGGTACATTTTTGTATTTGAATGTATCTATGTTACTCATGTCT
GTGTCAACTGGCAGACATGTGCTTTTGCATTTTAGAAGATCACTAG
AGGATGCCGGATGCTATGATTCAACAGTTATAGTATTGGAAAGGAC
CCATGTATAGACATGGACCTGCAAAAGGGAACCTTGTGGAAAGGCA
TCATGTTCTGGGTCCAGCCAGAGGGAAGAAAGCAAATGATGAAATC
CCAGATGGTCTCCGGGATCACCATTCCGAGCAGGGGCTGAAAGCCT
GTCCAAAGCTGGTAGAGACAAAAGCCCCTCTGCCTACCCAGGGTCA
TAATCAGACTCCTGCTCTGAGAATAAAATAGATGTTTGTGAAAGATG
Swine SP10 Promoter:
(SEQ ID NO: 12)
CAGTGGGTTAGGGATCTGGTGTTGCCGTGAGCTATGGTGTAGGTTG
CAGACATGGCTTGGATCCTTCGAAGGCCGGCAGCAACAGCTCCAAT
TAGACCCCTAGCCTGAGAATCTCCATATGCCACAGGCGTGGCCCTA
AAAGGACAAAAGACAAAATAAAGAAAGAAACAAAGAATCAAACAAG
AAATCACCAGCTACTCTCACCTCACTGTCAAGAATACTTTAAAAAG
AGAGTTTACGTTATTTTGGTGATAAGATTTTTTAAGGTAGGGCAGA
ACCCCAACACACCATTGTACACATGGTGAATTGGCCTTCACAGGTA
CAGTCTGCTCTAGGTTTGCCAGAGGGCCAACCTGCCTGCACAGGTG
CCATGAGGACCTCAGCACAGCCCATTGTGAGGAAACATGGATTGTT
TCTGAGGTTCTAGAATTTCCAGATGCTGTGGCTCAGCACTGGGAGC
TTCTGCTCATAGGTTTTCTTCACGGCTCTTGG
Swine ACE promoter:
(SEQ ID NO: 13)
ATTGACTGAGTGGGCCTCCTGGCTGGCATGGGGCAAGACAAATGTC
CCCCCCTTCCAAAGCTCCTAGTTCCCTGTGTCTTAATGCTGCTGTC
CTTGCCATCCAGTGGGGGCAGAAGTGGCCAGAGGTGGGGCAAAAGG
GCCAGGGCAGCAGTAGTGGACCCCAGTCAGAGGTCCCTGTGCCCAC
AGTGCCACTCTGCCTAACAGGGCAGTGCTGCAGGCAGGCTCCTCGC
GGCCCTGGCAGGAGGTGCTGAAGGACATGGTTGGCTCAGGGGCCCT
GGACGCTCAACCATTGCTCGACTACTTCCAGCCGGTCACACAGTGG
CTAGAGGAGCAGAACCAGCGGAGCGGCGATATCCTGGGCTGGCCAG
AATATCAGTGGCGCCCACCGATGCCCGACAATTACCCGGAGGGCAT
TGGTAAAGCCGGGAGGGAGACGGGGCGGGGCGCTACGGAGGGCTCT
GGGCTGGGCTCTGGCCCCAGGCCCTGGGTTCATGGCTCCAATCAGC
TTCTCCCAGCTGGGATAT
Swine CK-15 promoter:
(SEQ ID NO: 14)
CCGGGTTCGGAAGATTCCTCTCCCCACCCCCACCGCCCCCAAGGAG
CATTTTAGAGCCGTAGTTAGAAAGCAGAGTGCCATTTGCAGTGTGG
TAACCAAAAGCAGAGGAAATAGAGTTTCTTCATGTTCCAACTGCTG
TCTCTTTGGAATTCTTGTTCTCATTTATAAGCCTTAAAGTGCACGC
CTGTCTAAATGAGCTTTCTATGAATATATTTTTTTATATGCAATGA
ATTCATTTAAAACTTGGCTTTTAGGATATAGGAGCTGCTCTTAGAC
CATAGAGAGAGAATGATTTTAACAGCAAAAGGAGGGGGAACTTCGT
GTAGTTGCAATACTCAGTGAAGCCTGTGCCTGGGTCTTTTTGAAGT
TAGACCTCAAATATTGCATAGATGCTTTAGGAAGTGTGATCGGGGC
AGGGAATCAAGAGAAAGTAATTAACAGTTGATAAGGAGATTGGGGA
TGGAAGAGAATGATGGAACCAAAACAAGGAGCCAGGGCTCTTAGAA
GGGTAAGAGGAATTCATTAGAAAATGGGGCTCACAGAATAGAATGT
GGTTACAGATCTTACCCTCTCGTGTCCTTGGTGACATTTCCAAACA
ATTGCCAAACTAAACGAGGTTTTAGGATAGCTGAAATCAAGAGCCT
CTTCCCCATGTCCTTGAGAGGTGTCAAACTGAAGTTGCAAACATTT
TAGAAACTCTTTAGAAAAAGACCTTCAAAGAGAAACATGCAAAATG
AGTTTTCCATTTTAAAAAAATGATACGACTGTTTATCACGTATTTT
CCTATGAATGAAAGCAGTCCTAAGAAGAAAAAAGCATTTATCTGAG
TTGGTTTCGAAAGCGATTATAGCAATTAAAGTCCTCACGATGTGAA
ATACACATTTGAACATCATTCACAGCGCAATTTGGATGTTTATTTT
TGGTGCACCGAATAGTTTAAGTGTAAAGCAAAAATATTGGCACCAT
ATAACATAGGCAGCATTCTAGCATTAAACATAGCTTTCCCTCTTGA
AAAGTTTAAAATAAATTTCAGTGGTTTTCTTTTGTCCCCCCGA

All publications, patent applications, and patents set forth herein are hereby incorporated herein by reference for all purposes; in case of conflict, the instant specification controls.

### FURTHER DISCLOSURE

Embodiments include, for example:
1. A genetically modified sperm cell, the sperm cell comprising an X chromosome or a Y chromosome that comprises an exogenous sequence expressed by the sperm cell. The sequence may be cis-restricted, e.g., a cis-restricted transgene. Embodiments include those set forth in Example 11. 2. The sperm cell of 1 comprising the X chromosome. 3. The speini cell of 1 comprising the Y chromosome. 4. The sperm cell of any of 1-3 wherein the exogenous sequence encodes an electrostatic sorting agent. 5. The sperm cell of any of 1-3 wherein the exogenous sequence encodes a visualization agent. 6. The sperm cell of 0 wherein the visualization agent is chosen from the group consisting of fluorescent markers, dyes, DNA intercalating fluorescent dyes, calcium-activated dyes, and radiopaque agents. 7. The sperm cell of any of 1-3 wherein the exogenous sequence encodes a toxic molecule. 8. The sperm cell of 0 wherein the toxic molecule is chosen from the group consisting of a toxin, a nuclease, an apoptotic factor, and a fatal dominant negative. 9. The sperm cell of 0 wherein the toxic molecule is a toxin or a toxic gene product chosen from the group consisting of TOXIN gene, Barnase, diphtheria toxin A, thymidine kinase, and ricin toxin. 10. The sperm cell of any of 1-3 wherein the exogenous sequence encodes an antidote to a toxin. 11. The sperm cell of 0 wherein the antidote/toxin combination comprises Barnase/Barstar. 12. The sperm cell of any of 1-3 wherein the exogenous sequence encodes at least a portion of an antibody that binds an antigen. 13. The sperm cell of any of 1-3 wherein the exogenous sequence encodes an exogenous epitope. 14. The sperm cell of any of 1-3 wherein the exogenous sequence encodes biotin, avidin, or polyHis. 15. The sperm cell of any of 1-3 wherein the exogenous sequence effectively impairs sperm motility. 16. The sperm cell of any of 1-13 wherein the exogenous sequence is part of a fusion protein. 17. The sperm cell of 0 wherein the fusion protein is a fusion of the exogenous sequence and a sequence encoding a protein that localizes to a plasma membrane of the sperm—referring to the plasma membrane on an exterior of the sperm cell. 18. The sperm cell of 0 wherein the fusion protein is a fusion of the exogenous sequence and a sequence encoding a protein that selectively localizes to a portion of the sperm chosen from the group consisting of head, midpiece, tail, flagellum, endpiece, principal piece, and neck. 19. The sperm cell of any of 1-0 wherein the exogenous sequence is expressed on an exterior of the sperm cell. 20. The sperm cell of any of 1-0 wherein the exogenous sequence is expressed in an interior of the sperm cell. 21. A genetically modified animal producing a sperm cell as in any of 1-0. 22. Progeny of the animal of 0 expressing a sperm cell as in any of 1-0. 23. Sperm produced from an animal of 0 or 0. 24. A method of sorting sperm comprising creating an animal as in any of 1-0. 25. A method of sperm sorting comprising separating sperm comprising an X chromosome from sperm comprising a Y chromosome based on a presence of, or an absence of, at least one biologically expressed marker. 26. The method of 0 further comprising creating a founder animal that produces the sperm. 27. The method of 0 wherein the biologically expressed marker is chosen from the group consisting of fluorescent markers, dyes, DNA intercalating fluorescent dyes, calcium-activated dyes, and radiopaque agents, a color in a visible light wavelength, a color in a fluorescence wavelength, fluorescence, radiopacity, an exogenous epitope, a binding ligand, and at least a portion of an antibody. 28. The method of 0 or 0 comprising visualizing sperm with the marker. 29. The method of 0 or 0 comprising use of a FAC-SORT or a sperm selection device. 30. The method of 0 or 0 comprising binding the biologically expressed marker with a ligand that specifically binds the marker. 31. The method of 0 or 0 comprising binding the biologically expressed marker with a solid surface that comprises a ligand that specifically binds the marker, or binding a plurality of sperm to each other via crosslinkers that express a plurality of ligands that the specifically binds the expressed markers. 32. The method of 0 wherein the separation is based on sperm motility. 33. The method of 0 wherein the separation comprises a live/dead assay. 34. The method of 0 wherein the marker is chosen from the group consisting of a toxic substance and an antidote. 35. A system for sperm sorting comprising sperm comprising an X chromosome that expresses a marker, or sperm comprising a Y chromosome that expresses a marker, or sperm comprising an X chromosome that expresses a first marker in a mixture with sperm comprising a Y chromosome that expresses a second marker; and a binding moiety that selectively binds the marker, e.g., a ligand that has specific binding for the marker or a substance that binds substantially only the marker and not other sperm. 36. The system of 0 wherein the binding moiety is immobilized to a solid surface or a polymer. 37. The system of 0 wherein the binding moiety is attached to a toxic substance that damages sperm cells that are bound by the ligand. 38. The system of 0 wherein the binding moiety is a ligand is chosen from the group consisting of avidin, biotin, at least a portion of an antibody that binds the marker, a peptide that specifically binds the marker, an apatamer, and a nucleic acid that specifically binds the marker. 39. The system of 0 wherein the marker is for negative selection. Alternatively, wherein the marker is for positive selection. 40. A system for sperm sorting comprising sperm comprising an X chromosome that expresses a marker, or sperm comprising a Y chromosome that expresses a marker, or sperm comprising an X chromosome that expresses a first marker in a mixture with sperm comprising a Y chromosome that expresses a second marker; wherein the marker provides for separation by visualization. 41. A genetically modified livestock animal, the animal comprising an exogenous gene on an X chromosome or a Y chromosome, the gene expressing a marker in sperm of the animal. 42. The animal of 40 or 41, with the exogenous gene being under control of a gene expression element that is selectively activated in gametogenesis. 43. The animal of 0, with the exogenous gene being under control of an inducible promoter. 44. The animal of 0 or 0 wherein the chromosome is the Y chromosome. 45. The animal of 0 or 0 wherein the chromosome is the X chromosome. 46. The animal of 0-0 wherein the gene expression element comprises a promoter chosen from the group consisting of cyclin A1 promoter, Stra8, SP-10 promoter, a Stra8 promoter, C-Kit, ACE, and protamine. 47. The animal of 0-0 wherein the exogenous gene encodes a fusion of the factor and a microRNA. 48. An animal with sperm marked to indicate gender of a sex chromosome in each sperm. 49. The animal of 0 wherein the sex chromosome is an X and the X chromosome bears the marker. 50. The animal of 0 wherein the sex chromosome is an Y and the Y chromosome bears the marker. 51. A use of any of 1-50. A kit for making any of 1-50.

TABLE 1
Frequencies for recovery of colonies with HDR alleles

| | | | Mutation | | aa | Day 3% | | Bi-allelic |
|---|---|---|---|---|---|---|---|---|
| Reagent | ID | Species | type | nt change | change | HDR | HDR+ (%) | HDR+ (%) |
| TALEN | ssLDLR2.1^{a} | Pig ♀ | Ins/FS | 141(ins4) | 47ΔPTC | 38 | 55/184 (30) | 4/184 (2) |
| TALEN | ssDAZL3.1^{b} | Pig ♂ | Ins/FS | 173(ins4) | 57ΔPTC | 25 | 34/92 (37) | 8/92 (9) |
| TALEN | ssDAZL3.1^{Rep} | Pig ♂ | Ins/FS | 173(ins4) | 57ΔPTC | 30 | 42/124 (34) | 7/124 (6) |
| TALEN | ssAPC14.2^{b} | Pig ♂ | Ins/FS | 2703(ins4) | 902ΔPTC | 48 | 22/40 (55) | 4/40 (10) |
| TALEN | ssAPC14.2^{Rep} | Pig ♂ | Ins/FS | 2703(ins4) | 902ΔPTC | 50 | 57/96 (60) | 19/96 (20) |
| TALEN | ssAPC14.2^{td} | Pig ♂ | Ins/FS | 2703(ins4) | 902ΔPTC | 34 | 21/81 (26) | 1/81 (1) |
| TALEN | ssTp53 | Pig ♂ | Ins/FS | 463(ins4) | 154ΔPTC | 22 | 42/71 (59) | 12/71 (17) |
| TALEN | ssRAG2.1 | Pig ♂ | Ins/FS | 228(ins4) | 76ΔPTC | 47 | 32/77 (42) | 13/77 (17) |
| TALEN | btRosa1.2^{c} | Cow ♂ | Ins/mloxP | ins34 | NA | 45 | 14/22(64) | 7/22(32) |
| TALEN | ssSRY3.2 | Pig ♂ | Ins/mloxP | ins34 | NA | 30 | ND | ND |
| TALEN | ssKissR3.2 | Pig ♂ | Ins/FS | 322(ins6) | 107ΔPTC | 53 | 57/96 (59) | 17/96 (18) |
| | | | | 323(del2) | | | | |
| TALEN | btGDF83.1 | Cow ♂ | del/FS | 821 (del11) | FS | ~10 | 7/72 (10) | 2/72 (3) |
| TALEN | ssEIF4GI14.1 | Pig ♂ | SNPs | G2014A | N672D | 52 | 68/102(67) | 40/102 (39) |
| | | | | T2017C | L673F | | | |
| | | | | C2019T | | | | |
| TALEN | btGDF83.6N | Cow ♂ | SNPs | G938A | C313Y | 18 | 8/94 (9) | 3/94 (3) |
| | | | | T945C | | | | |
| TALEN | btGDF83.6N^{d} | Cow ♂ | SNP | G938A | C313Y | NA | 7/105 (7) | 2/105 (2) |
| TALEN | ssP65.8 | Pig ♂ | SNP | T1591C | S531P | 18 | 6/40 (15) | 3/40 (8) |
| TALEN | ssP65.8^{Rep} | Pig ♂ | SNP | T1591C | S531P | 7 | 9/63 (14) | 5/63 (8) |
| TALEN | ssGDF83.6^{d} | Pig ♂ | SNP | G938A | C313Y | NA | 3/90 (3) | 1/90 (1) |
| TALEN | caFecB6.1 | Goat ♂ | SNP | A747G | Q249R | 17 | 17/72 (24) | 3/72 (4) |
| TALEN | caCLPG1.1 | Goat ♂ | SNP | A→G | Non- | 4 | ND | ND |
| | | | | | coding | | | |
| CRISPR | ssP65 G1s | Pig ♂ | SNP | T1591C | S531P | 6 | 6/96 (6) | 2/96 (2) |
| CRISPR | ssP65 G2a | Pig ♂ | SNP | T1591C | S531P | 5 | 2/45 (4) | 0/45 |
| CRISPR | APC14.2 G1a | Pig ♂ | Ins/FS | 2703(ins4) | 902ΔPTC | 32 | ND | ND |

1. A genetically modified livestock animal, the animal producing sperm comprising a marker for selection of sperm with an X chromosome or sperm with a Y chromosome.

2. The animal of claim 1 wherein the X chromosome comprises an exogenous gene that encodes the marker.

3. The animal of claim 1 wherein the Y chromosome comprises an exogenous gene that encodes the marker.

4. The animal of claim 1 wherein the marker is expressed by an exogenous gene under control of an inducible promoter.

5. The animal of claim 1 wherein the marker is expressed by an exogenous gene under control of a gene expression element that is selectively activated in gametogenesis.

6. The animal of claim 5 wherein the gene expression element comprises a promoter chosen from the group consisting of cyclin A1 promoter, Stra8, SP-10 promoter, a Stra8 promoter, C-Kit, ACE, and protamine.

7. The animal of claim 1 wherein the marker is expressed by an exogenous gene that encodes a fusion of the marker and a microRNA.

8. The animal of claim 1 wherein the marker is expressed by an exogenous gene and the marker is selected from the group consisting of a selection marker, an electrostatic sorting agent, a visualization agent, and an exogenous antigen.

9. The animal of claim 8 wherein the marker is the visualization agent and is chosen from the group consisting of fluorescent markers, dyes, DNA intercalating fluorescent dyes, calcium-activated dyes, and radiopaque agents.

10. The animal of claim 8 wherein the marker is the selection marker.

11. The animal of claim 10 wherein the selection marker comprises a toxic molecule.

12. The animal of claim 11 wherein the toxic molecule is selected from the group consisting of a toxin, a nuclease, an apoptotic factor, and a fatal dominant negative.

13. The animal of claim 11 wherein the toxic molecule is a toxin or a toxic gene product chosen from the group consisting of TOXIN gene, Barnase, diphtheria toxin A, thymidine kinase, and ricin toxin.

14. The animal of claim 10 wherein the selection marker comprises an antidote to a toxin.

15. The animal of claim 8 wherein the marker is the antigen, with the antigen being selected from the group consisting of biotin, avidin, and polyHis.

16. The animal of claim 1 wherein the marker comprises a factor that impairs sperm motility.

17. The animal of claim 1 wherein the marker is expressed on an exterior of the sperm and has specific binding for a bimolecular factor.

18. The animal of claim 1 wherein the marker is part of a fusion protein that comprises a protein native to the sperm.

19. The animal of claim 18 wherein the protein native to the sperm is selected from the group consisting of exterior protein, interior protein, head, midpiece, tail, flagellum, endpiece, principal piece, and neck.

20. Sperm of the animal of claim 1.

21. A method of sperm sorting comprising separating sperm comprising an X chromosome from sperm comprising a Y chromosome based on a presence of, or an absence of, a biologically expressed marker.

22. The method of claim 21 wherein the biologically expressed marker is chosen from the group consisting of fluorescent markers, dyes, DNA intercalating fluorescent dyes, calcium-activated dyes, and radiopaque agents, a color in a visible light wavelength, a color in a fluorescence wavelength, fluorescence, radiopacity, an exogenous epitope, a binding ligand, and at least a portion of an antibody.

23. A system for sperm sorting comprising
sperm comprising an X chromosome that expresses a marker, or
sperm comprising a Y chromosome that expresses a marker, or
sperm comprising an X chromosome that expresses a first marker in a mixture with sperm comprising a Y chromosome that expresses a second marker; and
a binding moiety that selectively binds the marker or a device that uses the marker to sort the sperm.


SEQUENCE LISTING (14 sequences)

SEQ ID NO: 1 - DNA, 23 bp, Artificial Sequence
  Guide sequence
gctcaccaacggtctcctctcgg

SEQ ID NO: 2 - DNA, 22 bp, Artificial Sequence
  Guide sequence
gttgccagaggagagccccctg

SEQ ID NO: 3 - DNA, 92 bp, sus scrofa
gggcctctgggctcaccaacggtctcctctcgggggacgaagacttctcctccattgcggacatggacttctcagcccttctgagtcagatc

SEQ ID NO: 4 - DNA, 90 bp, Artificial Sequence
  HDR template
gggcctctgggctcaccaacggtctcctcccgggggacgaagacttctcctccattgcggacatggacttctcagcccttctgagtcaga

SEQ ID NO: 5 - DNA, 15 bp, Artificial Sequence
  TALEN
ctcctccattgcgga

SEQ ID NO: 6 - DNA, 15 bp, Artificial Sequence
  TALEN
cttctgagtcagatc

SEQ ID NO: 7 - DNA, 33 bp, Artificial Sequence
  TALEN
ggaagaagtatcagccatacagaaattctgggt

SEQ ID NO: 8 - DNA, 86 bp, sus scrofa
ccagatcgccaaatgcatggaagaagtatcagccattcatccctcccaggaagacagaaattctgggtcaaccacggagttgcact

SEQ ID NO: 9 - DNA, 23 bp, Artificial Sequence
  TALEN
gggagggtccttctgtctttaag

SEQ ID NO: 10 - DNA, 89 bp, Artificial Sequence
  HDR template
ccagatcgccaaagtcacggaagaagtatcagccattcatccctcccagtgaacttacagaaattctgggtcgaccacggagttgcact

SEQ ID NO: 11 - DNA, 1565 bp, Artificial Sequence
  EGFP cassette
tgtgtgtttgggaggagcttgtgtgtgtgagttgtgttttaagtttatttgcgtgtgagtacctttgggtttttgtgtgtgtctgtgtgtgtttgtgtgtgtataactgtgggtgactgtaagtgcacctgtgtgtttgtacgtgagtgtgtaagactgtgtgtgtgcacaagagcgtgtgtaggtgcacgtgttgtaggtgtgagaacacctgttgtgtttaggccatcagtcagcttggtcattgtttctaaggtagcatttatactttgttacctcaagtgggctctgggagtcaacagaagtcagaaaagctcagatccaagccccctttttctgacctcgagaccatggccaggaacgtcacgaaacggaaaaacaggtgccgcggacatcagaaggctatttacaagaaaaagtctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagtcgagatatgtcgacgacacaggaagggtgtcaggagaacgagcattcggctcggcacaggtacatttttgtatttgaatgtatctatgttactcatgtctgtgtcaactggcagacatgtgcttttgcattttagaagatcactagaggatgccggatgctatgattcaacagttatagtattggaaaggacccatgtatagacatggacctgcaaaagggaaccttgtggaaaggcatcatgttctgggtccagccagagggaagaaagcaaatgatgaaatcccagatggtctccgggatcaccattccgagcaggggctgaaagcctgtccaaagctggtagagacaaaagcccctctgcctacccagggtcataatcagactcctgctctgagaataaaatagatgtttgtgaaagatg

SEQ ID NO: 12 - DNA, 492 bp, sus scrofa
cagtgggttagggatctggtgttgccgtgagctatggtgtaggttgcagacatggcttggatccttcgaaggccggcagcaacagctccaattagacccctagcctgagaatctccatatgccacaggcgtggccctaaaaggacaaaagacaaaataaagaaagaaacaaagaatcaaacaagaaatcaccagctactctcacctcactgtcaagaatactttaaaaagagagtttacgttattttggtgataagattttttaaggtagggcagaaccccaacacaccattgtacacatggtgaattggccttcacaggtacagtctgctctaggtttgccagagggccaacctgcctgcacaggtgccatgaggacctcagcacagcccattgtgaggaaacatggattgtttctgaggttctagaatttccagatgctgtggctcagcactgggagcttctgctcataggttttcttcacggctcttgg

SEQ ID NO: 13 - DNA, 524 bp, sus scrofa
attgactgagtgggcctcctggctggcatggggcaagacaaatgtcccccccttccaaagctcctagttccctgtgtcttaatgctgctgtccttgccatccagtgggggcagaagtggccagaggtggggcaaaagggccagggcagcagtagtggaccccagtcagaggtccctgtgcccacagtgccactctgcctaacagggcagtgctgcaggcaggctcctcgcggccctggcaggaggtgctgaaggacatggttggctcaggggccctggacgctcaaccattgctcgactacttccagccggtcacacagtggctagaggagcagaaccagcggagcggcgatatcctgggctggccagaatatcagtggcgcccaccgatgcccgacaattacccggagggcattggtaaagccgggagggagacggggcggggcgctacggagggctctgggctgggctctggccccaggccctgggttcatggctccaatcagcttctcccagctgggatat

SEQ ID NO: 14 - DNA, 1055 bp, sus scrofa
ccgggttcggaagattcctctccccacccccaccgcccccaaggagcattttagagccgtagttagaaagcagagtgccatttgcagtgtggtaaccaaaagcagaggaaatagagtttcttcatgttccaactgctgtctctttggaattcttgttctcatttataagccttaaagtgcacgcctgtctaaatgagctttctatgaatatatttttttatatgcaatgaattcatttaaaacttggcttttaggatataggagctgctcttagaccatagagagagaatgattttaacagcaaaaggagggggaacttcgtgtagttgcaatactcagtgaagcctgtgcctgggtctttttgaagttagacctcaaatattgcatagatgctttaggaagtgtgatcggggcagggaatcaagagaaagtaattaacagttgataaggagattggggatggaagagaatgatggaaccaaaacaaggagccagggctcttagaagggtaagaggaattcattagaaaatggggctcacagaatagaatgtggttacagatcttaccctctcgtgtccttggtgacatttccaaacaattgccaaactaaacgaggttttaggatagctgaaatcaagagcctcttccccatgtccttgagaggtgtcaaactgaagttgcaaacattttagaaactctttagaaaaagaccttcaaagagaaacatgcaaaatgagttttccattttaaaaaaatgatacgactgtttatcacgtattttcctatgaatgaaagcagtcctaagaagaaaaaagcatttatctgagttggtttcgaaagcgattatagcaattaaagtcctcacgatgtgaaatacacatttgaacatcattcacagcgcaatttggatgtttatttttggtgcaccgaatagtttaagtgtaaagcaaaaatattggcaccatataacataggcagcattctagcattaaacatagctttccctcttgaaaagtttaaaataaatttcagtggttttcttttgtccccccga